(19) United States
(12) Patent Application Publication
(10) Pub. No.: US 2025/0320488 A1
(43) Pub. Date: Oct. 16, 2025

(54) METHODS OF PREPARING OLIGONUCLEOTIDE-DIRECTED COMBINATORIAL LIBRARIES

(71) Applicant: Insitro, Inc., South San Francisco, CA (US)

(72) Inventors: Divya KANICHAR, Los Altos, CA (US); Richard Edward WATTS, Walnut Creek, CA (US); Spurti Umesh AKKI, San Francisco, CA (US)

(73) Assignee: Insitro, Inc., South San Francisco, CA (US)

(21) Appl. No.: 19/175,408

(22) Filed: Apr. 10, 2025

Related U.S. Application Data
(60) Provisional application No. 63/633,292, filed on Apr. 12, 2024.

(51) Int. Cl. C12N 15/10

(52) U.S. Cl. CPC C12N 15/1068

(57) ABSTRACT

The present disclosure relates to precursor molecules of DNA-encoded compounds, and methods of preparing thereof. In some aspects, provided herein are methods of synthesizing DNA-encoded compounds, and libraries thereof, from precursor molecules and positional building blocks.

### CROSS-REFERENCE TO RELATED APPLICATIONS

This application claims priority to and the benefit of U.S. Provisional Application No. 63/633,292, filed on Apr. 12, 2024, the content of which is incorporated herein in its entirety.

### FIELD OF THE INVENTION

The present disclosure relates in some aspects to DNA-encoded compounds, and methods of preparing thereof. The disclosure also relates to methods of preparing libraries of DNA-encoded compounds and methods of using said libraries.

### BACKGROUND OF THE INVENTION

The field of combinatorial chemistry has made it possible to prepare complex libraries of candidate compounds in a single process. The members of these combinatorial libraries are synthesized by the addition of successive chemical subunits (i.e., building blocks) assembled on nucleic acids encoding their addition. The resulting library compounds may be tested for desired properties, including, but not limited to, their ability to bind to target molecules. Despite the success of many of these methods, existing methods of library synthesis have difficulty sorting libraries of oligonucleotides, resulting in slow partitioning of the library before synthesis of the encoded molecules on physical hybridization arrays. Thus, there is a need in the art for improved methods of synthesizing DNA-encoded compounds, including libraries of said compounds.

### SUMMARY OF THE INVENTION

Described herein are methods of synthesizing a DNA-encoded compound using a precursor molecule. The DNA-encoded compounds may be used to synthesize DNA-encoded libraries. The provided methods may improve the efficiency of DNA-encoded library synthesis.

In some aspects, provided herein is a method of synthesizing a DNA-encoded compound including an initial building block and at least one positional building block, the method including: (1) forming a precursor molecule, wherein the precursor molecule includes a DNA oligonucleotide including the initial building block at or near its 5′ terminus, wherein forming the precursor molecule includes: (a) providing a charged carrier, wherein the charged carrier includes the initial building block and an oligonucleotide sequence including a complementary region that is complementary to a portion on an RNA molecule, wherein the oligonucleotide sequence including the complementary region includes a codon that identifies the initial building block, (b) hybridizing the complementary region of the charged carrier to the RNA molecule,
(c) reverse transcribing the RNA molecule that is primed by the charged carrier to form a DNA-RNA heteroduplex, and (d) performing RNA hydrolysis on the DNA-RNA heteroduplex to form the precursor molecule; and (2) synthesizing the DNA-encoded compound using the precursor molecule and the at least one positional building block.

In any of the preceding embodiments, (i) the region that is complementary to a portion of the RNA molecule contains the codon; (ii) the complementary region of the RNA molecule contains a complementary sequence of the codon; and (iii) the codon on the charged carrier hybridizes to the complementary sequence in the complementary region of the RNA molecule.

In any of the preceding embodiments, (i) the charged carrier has the structure B-C-R, wherein B is the initial building block, R is the region that is complementary to a portion of the RNA molecule, and C is the codon that identifies the initial building block; (ii) the complementary region of the RNA molecule does not contain a complementary sequence of the codon.

In any of the preceding embodiments, the method further includes forming the charged carrier. In any of the preceding embodiments, forming the charged carrier includes: (i) immobilizing an uncharged carrier including a reactive moiety on a solid surface or synthesizing the uncharged carrier including the reactive moiety on the solid surface; (ii) reacting the initial building block with the reactive moiety to form an immobilized charged carrier; and (iii) releasing the immobilized charged carrier from the solid support to form the charged carrier.

In other aspects, provided herein is a method of synthesizing a DNA-encoded compound including an initial building block and at least one positional building block, the method including: (1) forming a precursor molecule, wherein the precursor molecule includes a DNA oligonucleotide including the initial building block at or near its 5′ terminus, wherein forming the precursor molecule includes: (a) providing an uncharged carrier, wherein the uncharged carrier includes a reactive moiety and an oligonucleotide sequence including a complementary region that is complementary to a portion on an RNA molecule, wherein the oligonucleotide sequence including the complementary region includes a codon that identifies the initial building block, (b) hybridizing the complementary region of the uncharged carrier to the RNA molecule, (c) reverse transcribing the RNA molecule primed by the uncharged carrier to form a DNA-RNA heteroduplex, (d) performing RNA hydrolysis on the DNA-RNA heteroduplex to form a DNA molecule including the reactive moiety, and (e) reacting the reactive moiety with the initial building block to form the precursor molecule; and (2) synthesizing the DNA-encoded compound using the precursor molecule and the at least one positional building block.

In any of the preceding embodiments, step (1)(e) includes: (i) ligating a second oligonucleotide to the 3′ terminus of the DNA molecule, wherein the second oligonucleotide includes a second reactive moiety at or near its 3′ terminus; and (ii) reacting the reactive moiety with the initial building block and reacting the second reactive moiety with a second initial building block to form the precursor molecule.

In any of the preceding embodiments, step (1)(e)(i) includes: (A) hybridizing a first splint to the 3′ terminus of the single-stranded DNA molecule to form a restriction site; (B) digesting the restriction site to form a truncated DNA molecule; (C) hybridizing or ligating a second splint to the 3′ terminus of the truncated DNA molecule, wherein the second splint forms an overhang; (D) hybridizing the second oligonucleotide to the overhang; and (E) ligating the second oligonucleotide to the truncated DNA molecule.

In any of the preceding embodiments, the 3′ end of the second oligonucleotide includes a hairpin structure including the reactive moiety.

In any of the preceding embodiments, (i) the region that is complementary to a portion of the RNA molecule contains the codon; (ii) the complementary region of the RNA molecule contains a complementary sequence of the codon; and (iii) the codon on the uncharged carrier hybridizes to the complementary sequence in the complementary region of the RNA molecule.

In any of the preceding embodiments, (i) the uncharged carrier has the structure M-C-R, wherein M is the reactive moiety, C is the codon that identifies the initial building block, and R is the region that is complementary to a portion of the RNA molecule; (ii) the complementary region of the RNA molecule does not contain a complementary sequence of the codon.

In any of the preceding embodiments, the precursor molecule further includes at least one non-coding region. In any of the preceding embodiments, the method further includes hybridizing a blocking oligonucleotide to the at least one non-coding region, wherein the blocking oligonucleotide does not hybridize to the codon.

In any of the preceding embodiments, the initial building block is not a nucleic acid or nucleic acid analog.

In any of the preceding embodiments, the second initial building block is not a nucleic acid or nucleic acid analog.

In any of the preceding embodiments, the initial building block is attached to the precursor molecule by a non-nucleotide linker.

In any of the preceding embodiments, the second initial building block is attached to the precursor molecule by a non-nucleotide linker.

In any of the preceding embodiments, the method further includes preparing the RNA molecule, wherein preparing the RNA molecule includes: (a) providing a double-stranded DNA template; (b) annealing a 5′ polymerase chain reaction (PCR) primer and 3′ PCR primer to the double-stranded DNA template, wherein at least one of the 5′ PCR primer and 3′ PCR primer include an RNA polymerase promoter sequence; (c) performing PCR to form an amplified DNA template including the RNA polymerase promoter sequence; and (d) transcribing the amplified DNA template to form the RNA molecule. In any of the preceding embodiments, the RNA polymerase promoter sequence is a T7 promoter sequence.

In some aspects, provided herein is a method of forming a DNA-encoded library including a plurality of DNA-encoded compounds, the method including forming a plurality of precursor molecules to synthesize the plurality of DNA-encoded compounds according to any of the preceding embodiments, wherein each of the plurality of precursor molecules include a different initial building block. In any of the preceding embodiments, the method further includes sorting the plurality of precursor molecules to a plurality of hybridization arrays, wherein, after sorting, each of the plurality of precursor molecules are further reacted with different positional building blocks corresponding to the hybridization arrays.

### BRIEF DESCRIPTION OF THE DRAWINGS

Representative embodiments of the invention are disclosed by reference to the following drawings. It should be understood that the embodiments depicted are not limited to the precise details shown.

FIG. 1A shows an exemplary schematic of a method of forming a precursor molecule with an initial building block at or near its 5′ terminus, where the precursor molecule is formed by providing a charged carrier including a codon that is complementary to a region on an RNA molecule.

FIGS. 1B-1C show an exemplary schematic of the initial steps a method of forming a precursor molecule with an initial building block at or near its 5′ terminus, where the precursor molecule is formed by providing a charged carrier and the RNA molecule includes a non-coding region at its 5′ terminus. The 5′ non-coding region of the RNA molecule and/or the non-coding region of the charged carrier may be blocked using blocking oligonucleotides (FIG. 1B), or the 5′ non-coding region of the RNA molecule may be digested via restriction digestion. Following block or digestion, the method of forming a precursor molecule may proceed according to the exemplary schematic shown in FIG. 1A.

FIG. 1D shows an exemplary schematic of a method of forming a precursor molecule with an initial building block at or near its 5′ terminus, where the precursor molecule is formed by providing a charged carrier including a conserved non-coding region that is complementary to a non-coding region on an RNA molecule.

FIG. 2 shows an exemplary schematic of a method of forming a precursor molecule with an initial building block at or near its 5′ terminus, where the precursor molecule is formed by providing an uncharged carrier.

FIG. 3 shows an exemplary schematic of a method of forming a bivalent precursor molecule with an initial building block at or near its 5′ and 3′ termini, where the precursor molecule is formed by providing a charged carrier.

FIG. 4 shows an exemplary schematic of a method of forming a bivalent precursor molecule with an initial building block at or near its 5′ and 3′ termini, where the precursor molecule is formed by providing an uncharged carrier.

FIG. 5 shows an exemplary schematic of a method of forming a charged carrier.

FIGS. 6A-6B show exemplary schematics of methods of forming a DNA-encoded compound using a positional building block and precursor molecule with an initial building block at or near its 5′ terminus (FIG. 6A), or a bivalent precursor molecule with initial building blocks at or near its 5′ and 3′ termini (FIG. 6B).

FIG. 7 shows the purity and yield of the reaction products at different stage of a method of forming a precursor molecule using a charged carrier.

FIG. 8 shows the purity and yield of the reaction products at different stage of a method of forming a bivalent precursor molecule using a charged carrier.

FIG. 9A shows the reaction yield (percentage conversion) of a bivalent precursor molecule when 480 Fmoc amino acids were used as initial building blocks.

FIG. 9B shows the reaction yield (percentage conversion) of a bivalent precursor molecule when 288 reductoids were used as initial building blocks.

### DETAILED DESCRIPTION OF THE INVENTION

Combinatorial chemistry using DNA-directed synthesis relies on the successive addition of building blocks (e.g., initial building blocks and/or positional building blocks) to precursor molecules to form DNA-encoded compounds. This synthesis is directed by codons in an oligonucleotide portion of the precursor molecules. DNA oligonucleotides used to encode DNA-encoded compounds are typically prepared initially as a complex library of oligonucleotides which include portions including different combinations of codons. Each codon is designed to direct the addition of a building block to the molecule.

The precursor molecules used to synthesize the DNA-encoded compounds include at least two portions: (1) an oligonucleotide including a plurality of codons and (2) at least one initial building block or a reactive moiety for addition of the at least one initial building block. The oligonucleotide portion directs the synthesis of an encoded region by sequence-specific hybridization of at least one of the codons to a capture oligonucleotide. The overall oligonucleotide portion also serves, downstream, to identify the encoded portion. For example, the oligonucleotide portion can be amplified by polymerase chain reaction (PCR) and then sequenced to identify the encoded portion.

To synthesize the DNA-encoded compounds, typically, capture oligonucleotides are immobilized on solid supports, such as on different pools of beads. The differently-labeled beads (including capture oligonucleotides) are then positioned in a spatially addressable array (i.e., a hybridization array). A complex mixture of precursor molecules is then added to the array. The precursor molecules then bind to capture oligonucleotides through sequence-specific hybridization of one or more of their codons to the appropriate capture oligonucleotide. After hybridization, those precursor molecules that did not have an appropriate codon to bind to a capture oligonucleotide can be washed away. The bound precursor molecules are then reacted with a positional building block and may then optionally be further sorted to another hybridization array for addition of a further positional building block.

The present disclose provides improved methods for synthesis of these DNA-encoded compounds, and DNA-encoded libraries thereof (that is, a library including a plurality of different DNA-encoded compounds), using a precursor molecule. The methods involve a reverse transcription (also referred to herein as “RT”) step to form the precursor molecules. Specifically, reverse transcription is used to install either an initial building block, or a reactive moiety for addition of the initial building block, to the precursor molecule. The use of reverse transcription in this manner avoids the need for a slow and inefficient sorting step during the preparation of the precursor molecules.

Thus, in some aspects, provided herein are methods of synthesizing a DNA-encoded compound including an initial building block and at least one positional building block, the method including: (1) forming a precursor molecule, wherein the precursor molecule includes a DNA oligonucleotide including the initial building block at or near its 5′ terminus, wherein forming the precursor molecule includes: (a) providing a charged carrier, wherein the charged carrier includes the initial building block and an oligonucleotide sequence including a complementary region that is complementary to a portion on an RNA molecule, wherein the oligonucleotide sequence including the complementary region includes a codon that identifies the initial building block, (b) hybridizing the complementary region of the charged carrier to the RNA molecule, (c) reverse transcribing the RNA molecule that is primed by the charged carrier to form a DNA-RNA heteroduplex, and (d) performing RNA hydrolysis on the DNA-RNA heteroduplex to form the precursor molecule. The precursor molecule may then be used to synthesize a DNA-encoded compound using the precursor molecule and the at least one positional building block.

Further provided herein are methods of synthesizing a DNA-encoded compound including an initial building block and at least one positional building block, the method including: (1) forming a precursor molecule, wherein the precursor molecule includes a DNA oligonucleotide including the initial building block at or near its 5′ terminus, wherein forming the precursor molecule includes: (a) providing an uncharged carrier, wherein the uncharged carrier includes a reactive moiety and an oligonucleotide sequence including a complementary region that is complementary to a portion on an RNA molecule, wherein the oligonucleotide sequence including the complementary region includes a codon that identifies the initial building block, (b) hybridizing the complementary region of the uncharged carrier to the RNA molecule, (c) reverse transcribing the RNA molecule primed by the uncharged carrier to form a DNA-RNA heteroduplex, (d) performing RNA hydrolysis on the DNA-RNA heteroduplex to form a DNA molecule including the reactive moiety, and (e) reacting the reactive moiety with the initial building block to form the precursor molecule. The precursor molecule may then be used to synthesize a DNA-encoded compound using the precursor molecule and the at least one positional building block. In some embodiments, the method further includes attaching a second initial building block at or near the 3′ terminus of the precursor molecule. For example, in some embodiments, step (1)(e) includes: (i) ligating a second oligonucleotide to the 3′ terminus of the DNA molecule, wherein the second oligonucleotide includes a second reactive moiety at or near its 3′ terminus; (ii) reacting the reactive moiety with the initial building block and reacting the second reactive moiety with a second initial building block to form the precursor molecule. In some embodiments, step (1)(e)(i) includes: (A) hybridizing a first splint to the 3′ terminus of the single-stranded DNA molecule to form a restriction site; (B) digesting the restriction site to form a truncated DNA molecule; (C) hybridizing or ligating a second splint to the 3′ terminus of the truncated DNA molecule, wherein the second splint forms an overhang; (D) hybridizing the second oligonucleotide to the overhang; (E) ligating the second oligonucleotide to the truncated DNA molecule.

In some embodiments, there is provided a method of forming a DNA-encoded library including a plurality of DNA-encoded compounds, the method including forming a plurality of precursor molecules to synthesize the plurality of DNA-encoded compounds according to any of the provided methods. In some embodiments, each of the plurality of precursor molecules include a different initial building block. In some embodiments, each of the plurality of precursor molecules include a different combination of unique codons in the oligonucleotide portion.

### Definitions

As used herein, the singular forms “a,” “an,” and “the” include the plural references unless the context clearly dictates otherwise.

Reference to “about” a value or parameter herein includes (and describes) variations that are directed to that value or parameter per se. For example, description referring to “about X” includes description of “X”.

It is understood that aspects and variations of the invention described herein encompass “comprising,” “consisting,” and/or “consisting essentially of” aspects and variations. The term “including” encompasses “comprising,” “consisting,” and “consisting essentially of” unless otherwise specified.

Unless otherwise noted, the term “hybridize,” “hybridizing,” and “hybridized” includes Watson-Crick base pairing, which includes guanine-cytosine and adenine-thymine (G-C and A-T) pairing for DNA and guanine-cytosine and adenine-uracil (G-C and A-U) pairing for RNA.

The terms “end” and “terminus”, in the context of describing the position of an initial building block described herein, are used synonymously to mean a position that is near the absolute end or absolute terminus of a precursor molecule or a charged carrier. For example, an initial building block at the 5′ terminus of a precursor may be described as being at a position at the “5′ end” or “5′ terminus” of the precursor molecule.

The “encoded region” of an DNA-encoded compound refers to the portion of the molecule that includes one or more building blocks, including initial building block(s) and/or positional building block(s).

The term “coding region” is used to describe a DNA oligonucleotide region of a DNA-encoded compound or precursor molecule that is used to identify the building block(s) (e.g., initial building block(s) and/or positional building block(s)) attached to the compound or molecule. For example, the coding region may be an oligonucleotide including a plurality of codons that encodes and directs the synthesis of the DNA-encoded compound, wherein the coding region determines which charged positional building blocks including anti-codons may hybridize to a codon of the coding region of DNA oligonucleotide of a precursor molecule or DNA-encoded compound, thereby synthesizing a DNA-encoded compound.

As used herein, a “plurality” of x means two or more of x. As used herein, a “multiplicity” of x means a plurality of x wherein each x are identical.

When a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The disclosures of all publications, patents, and patent applications referred to herein are each hereby incorporated by reference in their entireties. To the extent that any reference incorporated by reference conflicts with the instant disclosure, the instant disclosure shall control.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference. The section headings used herein are for organizational purposes only and are not to be construed as limiting the subject matter described.

### Precursor Molecules

The present disclosure provides methods of synthesizing DNA-encoded compounds, and libraries of DNA-encoded compounds, using precursor molecules. The precursor molecules include an oligonucleotide including an initial building block or a reactive moiety suitable for attaching an initial building block. In some embodiments, the precursor molecules include a first initial building block at the 5′ terminus and a second initial building block at the 3′ terminus (referred to herein as “bivalent precursor molecules”). The first and second initial building blocks may be the same building block or different building blocks. The precursor molecules may be formed using any of the methods described herein.

### DNA Oligonucleotide

The precursor molecules described herein include an oligonucleotide portion, i.e., a DNA oligonucleotide. The oligonucleotide includes a plurality of coding regions including codons. The oligonucleotide, in some embodiments, may further include non-coding regions. Non-coding regions can intersperse the codons of the coding region, and thus the non-coding regions would be within the coding region itself. The coding region, by function of the codons, may be used to identify the building blocks (e.g., the initial building block(s) and/or the positional building block(s)) of the precursor molecule, and the DNA-encoded compounds synthesized therefrom using a method described herein, during downstream analyses. In some embodiments, the coding region includes or is a DNA oligonucleotide.

The coding region may encode and direct the synthesis of a DNA-encoded compound from the precursor molecule. More specifically, the codons of the coding region direct the addition of successive positional building block(s) to the precursor molecule to eventually form the DNA-encoded compound. The codons of the coding region determine which anti-codons including may hybridize to the precursor molecule, and therefore which positional building block react with initial building blocks extending from initial building blocks, to synthesize the DNA-encoded compound. Additional description of coding region(s) and optional non-coding region(s) can be found in US 2020/0263163 A1 and US 2019/0169607 A1, which are hereby incorporated by reference in their entirety for all purposes.

The coding region including a plurality of codons may be partially or entirely single stranded. In some embodiments, the coding region is from about 1% to 100%, such as any of about 10% to about 75%, about 50% to about 100% or about 90% to about 100%, single stranded. In some embodiments, the coding region is at least partially single stranded.

In some embodiments, the precursor molecule includes a coding region including at least two codons, wherein the at least two codons correspond to and can be used to identify an initial building block in the precursor molecule or DNA-encoded compounds synthesized therefrom. In some embodiments, the coding region can be amplified by PCR to produce copies of the coding region and subsequently sequenced to determine the sequence of the coding region of the DNA oligonucleotide. The determined sequence can be used to identify an encoded region.

In some embodiments, the coding region of the precursor molecule is double stranded. In some embodiments, the coding region is single stranded. In some embodiments, the coding region of the precursor molecule is partially single stranded. In some embodiments, the coding region of the precursor molecule is partially double stranded.

The coding region of the precursor molecule includes a plurality of codons. The number of codons in the coding region determines how many unique anti-codons the coding region can specifically hybridize with during synthesis of the DNA-encoded compound from the precursor molecule. In some embodiments, the coding region includes between about 2 to about 21 codons, such as between any of about 2 to about 20 codons, about 5 to about 15 codons, and about 10 to about 21 codons. In some embodiments, the coding region includes less than about 21 codons, such as less than about any of about 20, 15, 5, or 3 codons. In some embodiments, the coding region includes about 2, 3, 4, 5, 6, 7, 8, 9, 10, 11, 12, 13, 14, 15, 16, 17, 18, 19, 20, or 21 codons. In some embodiments, the coding region includes between about 5 to about 20 codons. In some embodiments, two or more codons of the coding region may overlap with one another.

The codons used in DNA-encoded synthesis are typically longer than those found in nature. If a codon is less than about 6 nucleotides in length, the codon may not accurately direct synthesis of the encoded region of the DNA-encoded compound synthesized from the precursor molecule. If a codon is too long, such as more than about 50 nucleotides, the codon may become cross-reactive. Such cross reactivity interferes with the ability of the coding regions to accurately direct and identify the synthesis steps used to synthesize the coding region of the DNA oligonucleotide. Those skilled in the art of combinatorial chemistry can readily select an appropriate average codon length depending on the circumstances. In some embodiments, each codon of the plurality of codons of the coding region of the precursor molecule include between about 6 to about 50 nucleotides, such as between any of about 6 to about 20, about 8 to about 30, about 15 to about 25, and about 30 to about 50 nucleotides. In some embodiments, each codon of the precursor molecule includes less than about 50 nucleotides, such as less than any of about 45, 40, 35, 30, 25, 20, 15, 10, or 6 nucleotides. In some embodiments, each codon of the precursor molecule includes about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In some embodiments, each codon of the precursor molecule includes between about 8 and about 30 nucleotides. In some embodiments, each codon of the precursor molecule includes the same number of nucleotides. In some embodiments, each codon of the precursor molecule includes a different number of nucleotides. In some embodiments, a portion of the codons of the precursor molecule include the same number of nucleotides, and a portion of the codons of the precursor molecule include a different number of nucleotides.

In some embodiments, the codons of the coding region of the precursor molecule overlap. In some embodiments, at least two of the codons of the coding region of the precursor molecule overlap so as to be coextensive, provided that the overlapping codons only share from about 30% to 1% of the same nucleotides, including about 20% to 1%, including from about 10% to 2%. In some embodiments of the DNA oligonucleotide of the precursor molecule, the coding region is from about 30% to 100%, including about from 60% to 100%, including about from 80% to 100%, single stranded. In some embodiments, the DNA oligonucleotide includes at least two coding regions including at least one codon each, wherein at least two of the coding regions are adjacent. In some embodiments, the DNA oligonucleotide includes at least two coding regions, wherein the at least two coding regions are separated by regions of nucleotides that do not direct or record synthesis of an encoded portion of the synthesized DNA-encoded compound from the precursor molecule.

The oligonucleotide of the precursor molecule directs the synthesis of a DNA-encoded compound by selectively hybridizing to a complementary anti-codon. In some embodiments, each codon of a plurality of codons encodes for the addition of one positional building block of a plurality of positional building blocks. In some embodiments, a plurality of codons encodes for the addition of a plurality of building blocks.

The coding region of the precursor molecule can contain natural or unnatural nucleotides and combinations thereof. Suitable nucleotides include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Other suitable bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages. It is understood that an oligonucleotide is a polymer of nucleotides. In certain embodiments, the coding region of the precursor molecule does not have to contain contiguous bases. In certain embodiments, the coding region of the precursor molecule can be interspersed with linker moieties or non-nucleotide molecules.

In some embodiments, the coding region of the DNA oligonucleotide of the precursor molecule contains from about 5% to 100%, including from about 5% to about 50%, about 40% to about 80%, about 80% to 99%, about 90% to about 99%, or about 100% DNA nucleotides. In some embodiments, the coding region contains from about 5% to about 100%, including from about 5% to about 50%, about 40% to about 80%, about 80% to about 99%, about 90% to about 99%, or about 100% RNA nucleotides. In some embodiments, wherein the coding region includes a specified percentage of DNA nucleotides or RNA nucleotides, respectively, the remaining percentage includes RNA nucleotides of DNA nucleotides, respectively.

In some embodiments, the DNA oligonucleotide of the precursor molecule may further include a non-coding region or a plurality of non-coding regions. The term “non-coding region,” when present, refers to a region of the DNA oligonucleotide that does not correspond to any anti-coding nucleic acid used to synthesize a DNA-encoded compound on the encoded region of the DNA oligonucleotide of the precursor molecule. In some embodiments, non-coding regions are optional. In some embodiments, the DNA oligonucleotide contains from 1 to about 20 non-coding regions, including from 2 to about 9 non-coding regions, including from 2 to about 4 non-coding regions. In some embodiments, the non-coding regions contain from about 4 to about 50 nucleotides, including from about 12 to about 40 nucleotides, and including from about 8 to about 30 nucleotides. In some embodiments, one or more of the non-coding regions are double stranded, which reduces cross-hybridization.

The addition of non-coding regions in the precursor molecule can separate codons in the coding region to avoid or reduce cross-hybridization, because cross-hybridization would interfere with accurate encoding of a DNA-encoded compound synthesized from the DNA oligonucleotide of the precursor molecule. Further, the non-coding regions can add functionality to the coding region of the DNA oligonucleotide of the precursor molecule other than just hybridization with anti-codons or encoding. The non-coding regions may be interspersed with the codons of the coding region. For example, two codons of the coding region may be separated by a non-coding region. Thus, in some embodiments, a coding region of the precursor molecule includes one or more non-coding regions. In some embodiments, one or more of the non-coding regions can be modified with a label, such as a fluorescent label or a radioactive label. Such labels can facilitate the visualization or quantification of the DNA oligonucleotide of the precursor molecule. In some embodiments, one or more of the non-coding regions are modified with a functional group or tether which facilitates processing. In some embodiments, one or more of the non-coding regions of the precursor molecule are double stranded (e.g., “blocked”), which reduces cross-hybridization. Suitable non-coding regions are typically selected such that they do not interfere with PCR amplification of the nucleic acid portion of the DNA oligonucleotide of the precursor molecule (e.g., non-coding regions do not interfere with identification of the positional building blocks used to synthesize a DNA-encoded compound from the of the precursor molecule).

The coding region may also be associated with blocking oligonucleotides. The coding region including two or more codons and at least one non-coding region may be hybridized with a blocking oligonucleotide. In some embodiments, the blocking oligonucleotide spans a portion, or all of, a non-coding region. Hybridizing a blocking oligonucleotide to the non-coding region can reduce intra-strand interactions of the oligonucleotide and also reduce inter-strand hybridization events. Accordingly, in some embodiments, the majority or all of the non-coding regions in the oligonucleotide region of the precursor molecule can be associated with blocking oligonucleotides. In such an embodiment, it is preferred that at least some of the non-coding regions have identical sequences, allowing for identical blocking oligonucleotides to be used.

### Initial Building Blocks

The precursor molecules described herein include one or more initial building blocks. A “building block” as used herein is a chemical structural unit capable of being chemically linked to other chemical structural units (e.g., other building blocks). The precursor molecule includes an initial building block or a reactive moiety capable of accepting the initial building block at or near its 5′ terminus. In some embodiments, the precursor molecule may include a first initial building block at or near its 5′ terminus and a second initial building block at or near its 3′ terminus (i.e., a “bivalent precursor molecule”).

One means of installing the initial building block to the precursor molecule is by starting with a charged carrier including the initial building block and a DNA oligonucleotide sequence including a codon that identifies the initial building block. Alternatively, the initial building block may be reacted with a reactive moiety on the nascent precursor molecule. In such embodiments, it is the reactive moiety that is installed by way of an uncharged carrier.

The initial building block is, in preferred embodiments, not a nucleic acid or nucleic acid analog. The initial building block preferably includes one, two, or more reactive chemical groups that allow the initial building block to undergo a chemical reaction that links the initial building block to other building blocks, such as positional building blocks. In preferred embodiments, the initial building block is linked to other chemical structural units (e.g., other building blocks) by a covalent bond during formation of the DNA-encoded compound.

It is understood that part or all of the reactive chemical group of the initial building block may be lost when the initial building block undergoes a reaction to form a chemical linkage. For example, an initial building block in solution may have two reactive chemical groups. In this example, the initial building block in solution can be reacted with the reactive chemical group of a positional building block that is part of a chain of building blocks to increase the length of a chain, or extend a branch from the chain. When an initial building block is referred to in the context of a solution or as a reactant, then the initial building block will be understood to contain at least one reactive chemical group, but may contain two or more reactive chemical groups. When an initial building block is referred to the in the context of a polymer, oligomer, or molecule larger than the initial building block by itself, then the initial building block will be understood to have the structure of the initial building block as a (monomeric) unit of a larger molecule, even though one or more of the chemical reactive groups will have been reacted.

The types of molecules or compounds that can be used as initial building blocks are not generally limited, except that building blocks (including initial building blocks) are preferably not nucleic acids or nucleic acid analogs, and so long as the initial building block is capable of reacting together with another building block (e.g., a positional building block) to form a covalent bond. In some embodiments, the initial building block is not a nucleic acid or nucleic acid analog. Those skilled in the art readily understand that a wide variety of molecules can serve as initial and positional building blocks.

In some embodiments, the initial building block has one chemical reactive group to serve as a terminal unit. In some embodiments, the initial building block has 1, 2, 3, 4, 5, or 6 suitable reactive chemical groups. In some embodiments, a first initial building block, a second initial building block, and a positional building block each independently have 1, 2, 3, 4, 5, or 6 suitable reactive chemical groups. Suitable reactive chemical groups for initial building blocks include, a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a haloacetate, an aryl halide, an azide, a halide, a triflate, a diene, a dienophile, a boronic acid, an alkyne, and an alkene.

Any suitable coupling chemistry can be used to connect initial building blocks with other building blocks (e.g., positional building blocks), provided that the coupling chemistry is compatible with the presence of an oligonucleotide (which the building blocks are ultimately linked to).

Exemplary coupling chemistry includes, formation of amides by reaction of an amine, such as a DNA-linked amine, with an Fmoc-protected amino acid or other variously substituted carboxylic acids; formation of ureas by reaction of an amine, including a DNA-linked amine, with an isocyanate and another amine (ureation); formation of a carbamate by reaction of amine, including a DNA-linked amine, with a chloroformate (carbamoylation) and an alcohol; formation of a sulfonamide by reaction of an amine, including a DNA-linked amine, with a sulfonyl chloride; formation of a thiourea by reaction of an amine, including a DNA-linked amine, with thionocarbonate and another amine (thioureation); formation of an aniline by reaction of an amine, including a DNA-linked amine, with a heteroaryl halide (SNAr); formation of a secondary amine by reaction of an amine, including a DNA-linked amine, with an aldehyde followed by reduction (reductive animation); formation of a peptoid by acylation of an amine, including a DNA-linked amine, with chloroacetate followed by chloride displacement with another amine (an SN2 reaction); formation of an alkyne containing compound by acylation of an amine, including a DNA-linked amine, with a carboxylic acid substituted with an aryl halide, followed by displacement of the halide by a substituted alkyne (a Sonogashira reaction); formation of a biaryl compound by acylation of an amine, including a DNA-linked amine, with a carboxylic acid substituted with an aryl halide, followed by displacement of the halide by a substituted boronic acid (a Suzuki reaction); formation of a substituted triazine by reaction of an amine, including a DNA-linked amine, with a cyanuric chloride followed by reaction with another amine, a phenol, or a thiol (cyanurylation, Aromatic Substitution); formation of secondary amines by acylation of an amine including a DNA-linked amine, with a carboxylic acid substituted with a suitable leaving group like a halide or triflate, followed by displacement of the leaving group with another amine (SN2/SN1 reaction); and formation of cyclic compounds by substituting an amine with a compound bearing an alkene or alkyne and reacting the product with an azide, or alkene (Diels-Alder and Huisgen reactions). In certain embodiments of the reactions, the molecule reacting with the amine group, including a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a chloroacetate, an aryl halide, an alkene, halides, a boronic acid, an alkyne, and an alkene, has a molecular weight of from about 30 to about 1330 Daltons.

In some embodiments of the coupling reaction, the initial building block might be added by substituting an amine, including a DNA-linked amine, using any of the chemistries above with molecules bearing secondary reactive groups like amines, thiols, halides, boronic acids, alkynes, or alkenes. Then the secondary reactive groups can be reacted with building blocks bearing appropriate reactive groups. Exemplary secondary reactive group coupling chemistries include, acylation of the amine, including a DNA-linked amine, with an Fmoc-amino acid followed by removal of the protecting group and reductive animation of the newly deprotected amine with an aldehyde and a borohydride; reductive animation of the amine, including a DNA-linked amine, with an aldehyde and a borohydride followed by reaction of the now-substituted amine with cyanuric chloride, followed by displacement of another chloride from triazine with a thiol, phenol, or another amine; acylation of the amine, including a DNA-linked amine, with a carboxylic acid substituted by a heteroaryl halide followed by an SNAr reaction with another amine or thiol to displace the halide and form an aniline or thioether; and acylation of the amine, including a DNA-linked amine, with a carboxylic acid substituted by a haloaromatic group followed by substitution of the halide by an alkyne in a Sonogashira reaction; or substitution of the halide by an aryl group in a boronic ester-mediated Suzuki reaction.

In some embodiments, the coupling chemistries are based on suitable bond-forming reactions known in the art. See, for example, March, Advanced Organic Chemistry, fourth edition, New York: John Wiley and Sons (1992), Chapters 10 to 16; Carey and Sundberg, Advanced Organic Chemistry, Part B, Plenum (1990), Chapters 1-11; Goodnow er al, A Handbook for DNA-Encoded Chemistry: Theory and Applications for Exploring Chemical Space and Drug Discovery, New York: John Wiley and Sons (2014); and Coltman et al, Principles and Applications of Organotransition Metal Chemistry, University Science Books, Mill Valley, Calif. (1987), Chapters 13 to 20; each of which is incorporated herein by reference in its entirety.

In some embodiments, the initial building block can include one or more functional groups in addition to the reactive group or groups employed to attach (e.g., react) another building block (e.g., positional building block). One or more of these additional functional groups can be protected to prevent undesired reactions of these functional groups. Suitable protecting groups are known in the art for a variety of functional groups (Greene and Wuts, Protective Groups in Organic Synthesis, second edition, New York: John Wiley and Sons (1991), incorporated herein by reference in its entirety). Particularly useful protecting groups include Fmoc-groups, t-butyl esters and carbamates, acetals, trityl ethers and amines, acetyl esters, trimethylsilyl ethers, trichloroethyl ethers and esters and carbamates.

The type of initial building block is not generally limited, so long as the initial building block is compatible with one more reactive groups capable of forming a covalent bond with other building blocks (e.g., positional building blocks). In some embodiments, the initial building block is not a nucleic acid or nucleic acid analog.

Suitable initial building blocks include but are not limited to, a peptide, a saccharide, a glycolipid, a lipid, a proteoglycan, a glycopeptide, a sulfonamide, a nucleoprotein, a urea, a carbamate, a vinylogous polypeptide, an amide, a vinylogous sulfonamide peptide, an ester, a saccharide, a carbonate, a peptidylphosphonate, an azatides, a peptoid (oligo N-substituted glycine), an ether, an ethoxyformacetal oligomer, thioether, an ethylene, an ethylene glycol, disulfide, an arylene sulfide, a nucleotide, a morpholino, an imine, a pyrrolinone, an ethyleneimine, an acetate, a styrene, an acetylene, a vinyl, a phospholipid, a siloxane, an isocyanide, a isocyanate, and a methacrylate. In certain embodiments, the (BI)M or (B2)κ of formula (I) each independently represents a polymer of these building blocks having M or K units, respectively, including a polypeptide, a polysaccharide, a polyglycolipid, a polylipid, a polyproteoglycan, a polyglycopeptide, a polysulfonamide, a polynucleoprotein, a polyurea, a poly carbamate, a polyvinylogous polypeptide, a polyamide, a poly vinylogous sulfonamide peptide, a polyester, a polysaccharide, a polycarbonate, a polypeptidylphosphonate, a polyazatides, a polypeptoid (oligo N-substituted glycine), a polyethers, a polythoxyformacetal oligomer, a polythioether, a polyethylene, a polyethylene glycol, a poly disulfide, a polyarylene sulfide, a polynucleotide, a polymorpholino, a polyimine, a polypyrrolinone, a polyethyleneimine, a polyacetates, a polystyrene, a polyacetylene, a polyvinyl, a polyphospholipids, a polysiloxane, a polyisocyanide, a polyisocyanate, and a polymethacrylate. In certain embodiments, from about 50% to about 100%, including from about 60% to about 95%, and including from about 70% to about 90% of the building blocks have a molecular weight of from about 30 to about 500 Daltons, including from about 40 to about 350 Daltons, including from about 50 to about 200 Daltons.

It is understood that initial building blocks having two reactive groups would form a linear oligomeric or polymeric structure, or a linear non-polymeric molecule, containing each other building block (e.g., positional building blocks) as a unit. It is also understood that initial building blocks having three or more reactive groups could form molecules with branches at each building block having three or more reactive groups.

In some embodiments, the initial building block is attached to an oligonucleotide (e.g., a DNA oligonucleotide) by a linker. In some embodiments, the precursor molecule (e.g., the DNA oligonucleotide of the precursor molecule) includes one or more linkers. The term “linker” as used herein refers to a bifunctional molecule or a portion thereof, which attaches an initial building block to the DNA oligonucleotide of the precursor molecule. In some embodiments, the initial building block is attached to the linker by a covalent bond. In some embodiments, the precursor molecule includes a first linker and a second linker. In some embodiments, the first linker is different from the second linker.

Various commercially available linkers are amenable to the applications of the present methods. Example of linkers may include, but are not limited to, PEG (e.g., azido-PEG-NHS, or azido-PEG-amine, or di-azido-PEG), or an alkane acid chain moiety (e.g., 5-azidopentanoic acid, (S)-2-(azidomethyl)-1-Boc-pyrrolidine, 4-azidoaniline, or 4-azido-butan-1-oic acid N-hydroxysuccinimide ester); thiol-reactive linkers, such as those being PEG (e.g., SM(PEG)n NHS-PEG-maleimide), alkane chains (e.g., 3-(pyridin-2-yldisulfanyl)-propionic acid-Osu or sulfosuccinimidyl 6-(3′-[2-pyridyldithio]-propionamido)hexanoate)); and amidites for oligonucleotide synthesis, such as amino modifiers (e.g., 6-(trifluoroacetylamino)-hexyl-(2-cyanoethyl)-(N,N-diisopropyl)-phosphoramidite), thiol modifiers (e.g., 5-trityl-6-mercaptohexyl-1-[(2-cyanoethyl)-(N,N-diisopropyl)]-phosphoramidite, or chemically co-reactive pair modifiers (e.g., 6-hexyn-1-yl-(2-cyanoethyl)-(N,N-diisopropyl)-phosphoramidite, 3-dimethoxytrityloxy-2-(3-(3-propargyloxypropanamido)propanamido)propyl-1-0-succinoyl, long chain alkylamino CPG, or 4-azido-butan-1-oic acid N-hydroxysuccinimide ester)); and compatible combinations thereof.

Many kinds of chemistry are available for use in this invention (e.g., for reaction of an initial building block with another building block, such as a positional building block, or for creation of an initial building block with a reactive moiety). In theory, any chemical reaction could be used that does not chemically alter DNA. Reactions that are known to be sufficiently DNA compatible include but are not limited to: Wittig reactions, Heck reactions, homer-Wads-worth-Emmons reactions, Henry reactions, Suzuki couplings, Sonogashira couplings, Huisgen reactions, reductive aminations, reductive alkylations, peptide bond reactions, peptoid bond forming reactions, acylations, SN2 reactions, SNAr reactions, sulfonylations, ureations, thioureations, carbamoylations, formation of benzimidazoles, imidazolidinones, quinazolinones, isoindolinones, thiazoles, imidazopyridines, diol cleavages to form glyoxals, Diels-Alder reactions, indole-styrene couplings, Michael additions, alkene-alkyne oxidative couplings, aldol reactions, Fmoc-deprotections, trifluoroacetamide deprotections, Alloc-deprotections, Nvoc deprotections and Boc-deprotections. (See, Handbook for DNA-Encoded Chemistry (Goodnow R. A., Jr., Ed.) pp 319-347, 2014 Wiley, N.Y. March, Advanced Organic Chemistry, fourth edition, New York: John Wiley and Sons (1992), Chapters 10 to 16; Carey and Sundberg, Advanced Organic Chemistry, Part B, Plenum (1990), Chapters 1-11; and Coltman et al., Principles and Applications of Organotransition Metal Chemistry, University Science Books, Mill Valley, Calif. (1987), Chapters 13 to 20; each of which is incorporated herein by reference in its entirety.

### DNA-Encoded Compounds and Libraries of DNA-Encoded Compounds

The provided precursor molecules may be useful for the synthesis of a DNA-encoded compound using the precursor molecule and at least one positional building block. DNA-encoded compounds are synthesized by attaching successive positional building blocks to the initial building block at the 5′ terminus of the precursor molecule; or, in the case of a bivalent precursor molecule, the DNA-encoded compounds are synthesized by attaching successive positional building blocks to the initial building blocks at the 5′ and/or 3′ termini of the bivalent precursor molecule. The positional building blocks are added by first hybridizing a codon in the coding region of the precursor molecule to an anti-codon on a capture oligonucleotide of a hybridization array. After removing other unbound precursor molecules, the bound precursor molecule is then reacted with the positional building block of interest, increasing the encoded region by one building block. The precursor molecule containing the initial building block and a positional building block can then be sorted to another feature of the hybridization array, or a feature of another hybridization array, and another positional building block can be added. For example, a precursor molecule having the structure Bi-C1-C2-C3, where Bi is the initial building block, C1 is a codon identifying Bi, and C2 and C3 are different codons, is sorted to a first hybridization array (such as by hybridization of C2 to an anti-codon) to form B1-Bi-C1-C2-C3, where B1 is a first positional building block. The precursor molecule including B1-Bi- is then sorted to another hybridization (such as by hybridization of C3 to an anti-codon), where B2 can be added, forming B2-B1-Bi-C1-C2-C3. This process is repeated until the encoded region reaches the desired number of positional building blocks. Because the identity of the anti-codons are known at a particular feature of a hybridization array, and because the order a molecule is sorted to different features is known, the sequence of the coding region identifies precise structure of the encoded region. The sequence of the coding region can be determined readily, for example, by PCR amplification and then sequencing. The method may further include forming a DNA-encoded library by forming a plurality of precursor molecules to synthesize a plurality of DNA-encoded compounds.

### DNA Oligonucleotide

The DNA-encoded compounds of the present application are molecules including at least an oligonucleotide portion and an encoded portion. The encoded portion is synthesized by attaching building blocks to the precursor molecules described herein. These building blocks are generally and preferably non-nucleic acid molecules including a reactive moiety allowing for the attachment of the building block to the DNA-encoded compound and/or other building blocks. The first building block in the sequence is referred to as the initial building block. A DNA-encoded compound may include a plurality of initial building blocks, for example, when an initial building block is placed on each terminus of the precursor molecule used to synthesize the DNA-encoded compound. Attached to the initial building block(s) are positional building blocks. Once a desired number of different building blocks are added, the DNA-encoded compound can be assessed for desired properties, such as the ability to bind a target protein. Typically, a library of DNA-encoded compounds is synthesized, wherein the library includes a plurality of unique encoded portions. The library of DNA-encoded compounds is then screened or selected for desired properties, such as the ability of individual encoded portions to bind a target protein, and then those compounds exhibiting desirable properties are identified. The oligonucleotide portion of the DNA-encoded compound serves two functions. First, the oligonucleotide portion directs the synthesis of the encoded portion by combinatorial chemistry. Second, the oligonucleotide portion can be used to identify the encoded portion after screening or selection.

The DNA oligonucleotide of the DNA-encoded compound is the same DNA oligonucleotide as in the precursor molecule from which the DNA-encoded compound was synthesized. The DNA oligonucleotide includes a plurality of codons, and optionally non-coding regions. Non-coding regions may intersperse the codons of the coding region (and thus the non-coding regions would be within the broader coding region itself). The coding region of the DNA-encoded compound can be used to identify the encoded region, that is, the identity of the building blocks (e.g., the initial building blocks and/or the positional building blocks) of the DNA-encoded compound, during downstream analyses.

The coding region of a DNA oligonucleotide encodes and directs the synthesis of a DNA-encoded compound from the precursor molecule; the coding region determines which anti-codons including positional building blocks may hybridize to the precursor molecule and/or the resulting DNA-encoded compound, and therefore which positional building block react with initial building blocks and/or positional building extending from initial building blocks, to synthesize the DNA-encoded compound. Additional description of coding region(s) and optional non-coding region(s) can be found in US 2020/0263163 A1 and US 2019/0169607 A1, which are hereby incorporated by reference in their entirety for all purposes.

The coding region of the DNA-encoded compound may be partially or entirely single stranded. In some embodiments, the coding region contains from about 1% to 100%, such as any of about 50% to about 100% or about 90% to about 100%, single stranded DNA oligonucleotide. In some embodiments, the coding region is at least partially single stranded.

In some embodiments, the DNA-encoded compound includes a coding region including at least two codons, wherein the at least two codons correspond to and can be used to identify a positional building block in the DNA oligonucleotide or molecules synthesized therefrom. In some embodiments, the coding region of the DNA-encoded compound can be amplified by PCR to produce copies of the coding region and the original and/or copies can be sequenced to determine the sequence of the coding region of the DNA oligonucleotide. The determined sequence can be used to identify the positional building blocks of the DNA-encoded compound. In some embodiments, the sequence of the coding regions of the DNA-encoded compound can be correlated to the series of combinatorial chemistry steps used to synthesize the encoded region (such as the initial building blocks and positional building blocks extending therefrom).

In some embodiments, the coding region of the DNA-encoded compound is double stranded. In some embodiments, the coding region of the DNA-encoded compound is single stranded. In some embodiments, the coding region of the DNA-encoded compound is partially single stranded. The coding region of the DNA-encoded compound includes a plurality of codons. The number of codons in the coding region of the of the DNA-encoded compound determines how many unique anti-codons (e.g., anti-codons of charged positional building blocks) the coding region can specifically hybridize with. If the number of codons of the DNA-encoded compound is below 2, the encoded portion may be too small to be practical. If the number of codons is too far above 20, synthetic inefficiencies may interfere with accurate synthesis. Thus, the number of codons of the DNA-encoded compound is typically a value between these lower and upper quantities. In some embodiments, the coding region of the DNA-encoded compound includes between about 2 to about 21 codons, such as between any of about 2 to about 20 codons, about 5 to about 15 codons, and about 10 to about 21 codons. In some embodiments, the coding region of the DNA-encoded compound includes less than about 21 codons, such as less than about any of about 20, 15, 5, or 3 codons. In some embodiments, the coding region includes about 2, 3, 4, 5, 6, 7, 8, 9, 10, 11, 12, 13, 14, 15, 16, 17, 18, 19, 20, or 21 codons. In some embodiments, the coding region includes between about 5 to about 20 codons. In some embodiments, the codons of the coding regions of the DNA-encoded compound may overlap with one another.

DNA-encoded synthesis uses the above-described codons to hybridize with anti-codons (e.g., anti-codons attached to charged positional building blocks). The codons used in DNA-encoded synthesis are typically longer than those used in nature (i.e., those which are scanned by a ribosome along an mRNA). If a codon is less than about 6 nucleotides in length, the codon may not accurately direct synthesis of the encoded region. If a codon is too long, such as more than about 50 nucleotides, the codon may become cross-reactive. Such cross reactivity would interfere with the ability of the coding regions to accurately direct and identify the synthesis steps used to synthesize the coding region of the DNA oligonucleotide. Thus, in some embodiments, each codon of the plurality of codons of a coding region of the DNA-encoded compound includes between about 6 to about 50 nucleotides, such as between any of about 6 to about 20, about 8 to about 30, about 15 to about 25, and about 30 to about 50 nucleotides. In some embodiments, each codon of the DNA-encoded compound includes less than about 50 nucleotides, such as less than any of about 45, 40, 35, 30, 25, 20, 15, 10, or 6 nucleotides. In some embodiments, each codon of the DNA-encoded compound includes about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In some embodiments, each codon of the DNA-encoded compound includes between about 8 and about 30 nucleotides.

In some embodiments, the codons of the coding region of the DNA-encoded compound overlap. In some embodiments, at least two of the codons of the coding region of the DNA-encoded compound overlap so as to be coextensive, provided that the overlapping codons only share from about 30% to 1% of the same nucleotides, including about 20% to 1%, including from about 10% to 2%. In some embodiments of the DNA oligonucleotide of the of the DNA-encoded compound, the coding region is from about 30% to 100%, including about from 60% to 100%, including about from 80% to 100%, single stranded. In some embodiments, the DNA oligonucleotide includes at least two coding regions including at least one codon each, wherein at least two of the coding regions are adjacent. In some embodiments, the DNA oligonucleotide includes at least two coding regions, wherein the at least two coding regions are separated by regions of nucleotides that do not direct or record synthesis of an encoded portion of the synthesized DNA-encoded compound.

The DNA oligonucleotide of the DNA-encoded compound may direct the synthesis of an encoded molecule by selectively hybridizing to a complementary anti-codon including a building block (i.e., a charged positional building block). In some embodiments, a codon of the coding region of the DNA-encoded compound is unique to (e.g., corresponds to) the identity of a positional building block that is attached to a reactive site of the DNA oligonucleotide. In some embodiments, a charged positional building block includes a building block and at least one corresponding anti-codon which hybridizes with at least one of the plurality of codons in the coding region of the DNA-encoded compound.

In some embodiments, at least one codon in the coding region of the DNA oligonucleotide of the DNA-encoded compound encodes the addition of a positional building block (e.g., a positional building block of a charged positional building block) to a reactive site. In some embodiments, at least one codon encodes the addition of a positional building block to the reactive site. In some embodiments, at least one codon of a plurality of codons of the DNA-encoded compound encodes for the addition of one positional building block of a plurality of positional building blocks. In some embodiments, each codon of a plurality of codons of the DNA-encoded compound encodes for the addition of one positional building block of a plurality of positional building blocks. In some embodiments, a plurality of codons of the DNA-encoded compound encodes for the addition of a plurality of positional building blocks.

The coding region of the DNA-encoded compound can contain natural and unnatural nucleotides. Suitable nucleotides include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Other suitable bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages. It is understood that an oligonucleotide is a polymer of nucleotides. In certain embodiments, the coding region does not have to contain contiguous bases. In certain embodiments, the coding region can be interspersed with linker moieties or non-nucleotide molecules.

In some embodiments, the coding region of the DNA oligonucleotide of the DNA-encoded compound contains from about 5% to 100%, including from about 5% to about 50%, about 40% to about 80%, about 80% to 99%, about 90% to about 99%, or about 100% DNA nucleotides. In some embodiments, the coding region contains from about 5% to about 100%, including from about 5% to about 50%, about 40% to about 80%, about 80% to about 99%, about 90% to about 99%, or about 100% RNA nucleotides. In some embodiments, wherein the coding region includes a specified percentage of DNA nucleotides or RNA nucleotides, respectively, the remaining percentage includes RNA nucleotides of DNA nucleotides, respectively.

In some embodiments, the DNA oligonucleotide may further include a non-coding region or a plurality of non-coding regions. The term “non-coding region,” when present, refers to a region of the DNA oligonucleotide that does not correspond to any anti-coding nucleic acid used to synthesize a DNA-encoded compound on the encoded region of the DNA oligonucleotide. In some embodiments, non-coding regions are optional. In some embodiments, the oligonucleotide G contains from 1 to about 20 non-coding regions, including from 2 to about 9 non-coding regions, including from 2 to about 4 non-coding regions. In some embodiments, the non-coding regions contain from about 4 to about 50 nucleotides, including from about 12 to about 40 nucleotides, and including from about 8 to about 30 nucleotides. In some embodiments, one or more of the non-coding regions are double stranded, which reduces cross-hybridization.

The addition of non-coding regions can separate codons in the coding region to avoid or reduce cross-hybridization, because cross-hybridization would interfere with accurate encoding of a DNA-encoded compound synthesized from the DNA oligonucleotide. Further, the non-coding regions can add functionality to the coding region of the DNA oligonucleotide other than just hybridization with anti-codons or encoding. The non-coding regions may be interspersed with the codons of the coding region. For example, two codons of the coding region may be separated by a non-coding region. Thus, in some embodiments, a coding region includes one or more non-coding regions. In some embodiments, one or more of the non-coding regions can be modified with a label, such as a fluorescent label or a radioactive label. Such labels can facilitate the visualization or quantification of the DNA oligonucleotide. In some embodiments, one or more of the non-coding regions are modified with a functional group or tether which facilitates processing. In some embodiments, one or more of the non-coding regions are double stranded (e.g., “blocked”), which reduces cross-hybridization. Suitable non-coding regions are typically selected that do not interfere with PCR amplification of the nucleic acid portion of the DNA oligonucleotide (e.g., non-coding regions do not interfere with identification of the building blocks used to synthesize a DNA-encoded compound).

### Building Blocks

The DNA-encoded described herein include one or more building blocks (e.g., one or more initial building blocks and/or one or more positional building blocks). The DNA-encoded compounds include an initial building block, which is part of the precursor molecule used to synthesize the DNA-encoded compound. The initial building block may be introduced to the precursor molecule by use of a charged carrier including the initial building block and an oligonucleotide sequence including a codon that identifies the initial building block. The initial building block may also be an individual initial building block capable of reacting with a reactive moiety on an uncharged carrier including an oligonucleotide sequence including a codon that identifies the initial building block.

In preferred embodiments, the initial building block is not a nucleic acid or nucleic acid analog. In preferred embodiments, the positional building block(s) are not nucleic acids or nucleic acid analogs. In some embodiments, a building block has one, two, or more reactive chemical groups that allow the building block to undergo a chemical reaction that links the building block to other chemical structural units (e.g., other chemical structural units present in other building block, such as positional building blocks). In some embodiments, the building block is linked to other chemical structural units (e.g., other building blocks) by a covalent bond.

It is understood that part or all of the reactive chemical group of a building block (e.g., initial building block or positional building block) may be lost when the building block undergoes a reaction to form a chemical linkage. For example, a building block in solution may have two reactive chemical groups. In this example, the building block in solution can be reacted with the reactive chemical group of a building block that is part of a chain of building blocks to increase the length of a chain, or extend a branch from the chain. When a building block is referred to in the context of a solution or as a reactant, then the building block will be understood to contain at least one reactive chemical group, but may contain two or more reactive chemical groups. When a building block is referred to the in the context of a polymer, oligomer, or molecule larger than the building block by itself, then the building block will be understood to have the structure of the building block as a (monomeric) unit of a larger molecule, even though one or more of the chemical reactive groups will have been reacted.

The types of molecule or compound that can be used as a building block (e.g., initial building block or positional building block) are not generally limited, so long as one building block is capable of reacting together with another building block to form a covalent bond. In some embodiments, the building block is not a nucleic acid or nucleic acid analog. In some embodiments, the building block is a chemical structural unit.

In some embodiments, the building block (e.g., initial building block or positional building block) has one chemical reactive group to serve as a terminal unit. In some embodiments, the building block has 1, 2, 3, 4, 5, or 6 suitable reactive chemical groups. In some embodiments, a first initiator building block, a second initiator building block, and a polymer building block each independently have 1, 2, 3, 4, 5, or 6 suitable reactive chemical groups. Suitable reactive chemical groups for building blocks include, a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a haloacetate, an aryl halide, an azide, a halide, a triflate, a diene, a dienophile, a boronic acid, an alkyne, and an alkene.

Any coupling chemistry can be used to connect building blocks (e.g., initial building block(s) or positional building block(s)), provided that the coupling chemistry is compatible with the presence of an oligonucleotide.

Exemplary coupling chemistry includes, formation of amides by reaction of an amine, such as a DNA-linked amine, with an Fmoc-protected amino acid or other variously substituted carboxylic acids; formation of ureas by reaction of an amine, including a DNA-linked amine, with an isocyanate and another amine (ureation); formation of a carbamate by reaction of amine, including a DNA-linked amine, with a chloroformate (carbamoylation) and an alcohol; formation of a sulfonamide by reaction of an amine, including a DNA-linked amine, with a sulfonyl chloride; formation of a thiourea by reaction of an amine, including a DNA-linked amine, with thionocarbonate and another amine (thioureation); formation of an aniline by reaction of an amine, including a DNA-linked amine, with a heteroaryl halide (SNAr); formation of a secondary amine by reaction of an amine, including a DNA-linked amine, with an aldehyde followed by reduction (reductive animation); formation of a peptoid by acylation of an amine, including a DNA-linked amine, with chloroacetate followed by chloride displacement with another amine (an SN2 reaction); formation of an alkyne containing compound by acylation of an amine, including a DNA-linked amine, with a carboxylic acid substituted with an aryl halide, followed by displacement of the halide by a substituted alkyne (a Sonogashira reaction); formation of a biaryl compound by acylation of an amine, including a DNA-linked amine, with a carboxylic acid substituted with an aryl halide, followed by displacement of the halide by a substituted boronic acid (a Suzuki reaction); formation of a substituted triazine by reaction of an amine, including a DNA-linked amine, with a cyanuric chloride followed by reaction with another amine, a phenol, or a thiol (cyanurylation, Aromatic Substitution); formation of secondary amines by acylation of an amine including a DNA-linked amine, with a carboxylic acid substituted with a suitable leaving group like a halide or triflate, followed by displacement of the leaving group with another amine (SN2/SN1 reaction); and formation of cyclic compounds by substituting an amine with a compound bearing an alkene or alkyne and reacting the product with an azide, or alkene (Diels-Alder and Huisgen reactions). In certain embodiments of the reactions, the molecule reacting with the amine group, including a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a chloroacetate, an aryl halide, an alkene, halides, a boronic acid, an alkyne, and an alkene, has a molecular weight of from about 30 to about 1330 Daltons.

In some embodiments of the coupling reaction, the building block (e.g., initial building block or positional building block) might be added by substituting an amine, including a DNA-linked amine, using any of the chemistries above with molecules bearing secondary reactive groups like amines, thiols, halides, boronic acids, alkynes, or alkenes. Then the secondary reactive groups can be reacted with building blocks bearing appropriate reactive groups. Exemplary secondary reactive group coupling chemistries include, acylation of the amine, including a DNA-linked amine, with an Fmoc-amino acid followed by removal of the protecting group and reductive animation of the newly deprotected amine with an aldehyde and a borohydride; reductive animation of the amine, including a DNA-linked amine, with an aldehyde and a borohydride followed by reaction of the now-substituted amine with cyanuric chloride, followed by displacement of another chloride from triazine with a thiol, phenol, or another amine; acylation of the amine, including a DNA-linked amine, with a carboxylic acid substituted by a heteroaryl halide followed by an SNAr reaction with another amine or thiol to displace the halide and form an aniline or thioether; and acylation of the amine, including a DNA-linked amine, with a carboxylic acid substituted by a haloaromatic group followed by substitution of the halide by an alkyne in a Sonogashira reaction; or substitution of the halide by an aryl group in a boronic ester-mediated Suzuki reaction.

In some embodiments, the coupling chemistries are based on suitable bond-forming reactions known in the art. See, for example, March, Advanced Organic Chemistry, fourth edition, New York: John Wiley and Sons (1992), Chapters 10 to 16; Carey and Sundberg, Advanced Organic Chemistry, Part B, Plenum (1990), Chapters 1-11; Goodnow er al, A Handbook for DNA-Encoded Chemistry: Theory and Applications for Exploring Chemical Space and Drug Discovery, New York: John Wiley and Sons (2014); and Coltman et al, Principles and Applications of Organotransition Metal Chemistry, University Science Books, Mill Valley, Calif. (1987), Chapters 13 to 20; each of which is incorporated herein by reference in its entirety.

In some embodiments, the building block (e.g., initial building block or positional building block) can include one or more functional groups in addition to the reactive group or groups employed to attach (e.g., react) a building block. One or more of these additional functional groups can be protected to prevent undesired reactions of these functional groups. Suitable protecting groups are known in the art for a variety of functional groups (Greene and Wuts, Protective Groups in Organic Synthesis, second edition, New York: John Wiley and Sons (1991), incorporated herein by reference in its entirety). Particularly useful protecting groups include Fmoc-groups, t-butyl esters and carbamates, acetals, trityl ethers and amines, acetyl esters, trimethylsilyl ethers, trichloroethyl ethers and esters and carbamates.

The type of building block is not generally limited, so long as the building block is compatible with one more reactive groups capable of forming a covalent bond with other building blocks. In some embodiments, the building block is not a nucleic acid or nucleic acid analog.

Suitable building blocks (e.g., initial building block or positional building block) include but are not limited to, a peptide, a saccharide, a glycolipid, a lipid, a proteoglycan, a glycopeptide, a sulfonamide, a nucleoprotein, a urea, a carbamate, a vinylogous polypeptide, an amide, a vinylogous sulfonamide peptide, an ester, a saccharide, a carbonate, a peptidylphosphonate, an azatides, a peptoid (oligo N-substituted glycine), an ether, an ethoxyformacetal oligomer, thioether, an ethylene, an ethylene glycol, disulfide, an arylene sulfide, a nucleotide, a morpholino, an imine, a pyrrolinone, an ethyleneimine, an acetate, a styrene, an acetylene, a vinyl, a phospholipid, a siloxane, an isocyanide, a isocyanate, and a methacrylate. In certain embodiments, the (BI)M or (B2)κ of formula (I) each independently represents a polymer of these building blocks having M or K units, respectively, including a polypeptide, a polysaccharide, a polyglycolipid, a polylipid, a polyproteoglycan, a polyglycopeptide, a polysulfonamide, a polynucleoprotein, a polyurea, a poly carbamate, a polyvinylogous polypeptide, a polyamide, a poly vinylogous sulfonamide peptide, a polyester, a polysaccharide, a polycarbonate, a polypeptidylphosphonate, a polyazatides, a polypeptoid (oligo N-substituted glycine), a polyethers, a polythoxyformacetal oligomer, a polythioether, a polyethylene, a polyethylene glycol, a poly disulfide, a polyarylene sulfide, a polynucleotide, a polymorpholino, a polyimine, a polypyrrolinone, a polyethyleneimine, a polyacetates, a polystyrene, a polyacetylene, a polyvinyl, a polyphospholipids, a polysiloxane, a polyisocyanide, a polyisocyanate, and a polymethacrylate. In certain embodiments, from about 50% to about 100%, including from about 60% to about 95%, and including from about 70% to about 90% of the building blocks have a molecular weight of from about 30 to about 500 Daltons, including from about 40 to about 350 Daltons, including from about 50 to about 200 Daltons.

It is understood that building blocks (e.g., initial building block(s) or positional building block(s)) having two reactive groups would form a linear oligomeric or polymeric structure, or a linear non-polymeric molecule, containing each building block as a unit. It is also understood that building blocks having three or more reactive groups could form molecules with branches at each building block having three or more reactive groups.

In some embodiments, the first building block is attached to an oligonucleotide G by a linker. In some embodiments, the oligonucleotide G includes a first linker and a second linker. In some embodiments, the first linker is different from the second linker. In some embodiments, the oligonucleotide G includes two or more linkers. The term “linker” as used herein refers to a bifunctional molecule or a portion thereof, which attaches a building block to the oligonucleotide G. In some embodiments, the building block is attached to the linker by a covalent bond.

Various commercially available linkers are amenable to the applications of the present methods. Example of linkers may include, but are not limited to, PEG (e.g., azido-PEG-NHS, or azido-PEG-amine, or di-azido-PEG), or an alkane acid chain moiety (e.g., 5-azidopentanoic acid, (S)-2-(azidomethyl)-1-Boc-pyrrolidine, 4-azidoaniline, or 4-azido-butan-1-oic acid N-hydroxysuccinimide ester); thiol-reactive linkers, such as those being PEG (e.g., SM(PEG)n NHS-PEG-maleimide), alkane chains (e.g., 3-(pyridin-2-yldisulfanyl)-propionic acid-Osu or sulfosuccinimidyl 6-(3′-[2-pyridyldithio]-propionamido)hexanoate)); and amidites for oligonucleotide synthesis, such as amino modifiers (e.g., 6-(trifluoroacetylamino)-hexyl-(2-cyanoethyl)-(N,N-diisopropyl)-phosphoramidite), thiol modifiers (e.g., 5-trityl-6-mercaptohexyl-1-[(2-cyanoethyl)-(N,N-diisopropyl)]-phosphoramidite, or chemically co-reactive pair modifiers (e.g., 6-hexyn-1-yl-(2-cyanoethyl)-(N,N-diisopropyl)-phosphoramidite, 3-dimethoxytrityloxy-2-(3-(3-propargyloxypropanamido)propanamido)propyl-1-0-succinoyl, long chain alkylamino CPG, or 4-azido-butan-1-oic acid N-hydroxysuccinimide ester)); and compatible combinations thereof.

Many kinds of chemistry are available for use in this invention (e.g., for reaction of a building block with another building block). In theory, any chemical reaction could be used that does not chemically alter DNA. Reactions that are known to be sufficiently DNA compatible include but are not limited to: Wittig reactions, Heck reactions, homer-Wads-worth-Emmons reactions, Henry reactions, Suzuki couplings, Sonogashira couplings, Huisgen reactions, reductive aminations, reductive alkylations, peptide bond reactions, peptoid bond forming reactions, acylations, SN2 reactions, SNAr reactions, sulfonylations, ureations, thioureations, carbamoylations, formation of benzimidazoles, imidazolidinones, quinazolinones, isoindolinones, thiazoles, imidazopyridines, diol cleavages to form glyoxals, Diels-Alder reactions, indole-styrene couplings, Michael additions, alkene-alkyne oxidative couplings, aldol reactions, Fmoc-deprotections, trifluoroacetamide deprotections, Alloc-deprotections, Nvoc deprotections and Boc-deprotections. (See, Handbook for DNA-Encoded Chemistry (Goodnow R. A., Jr., Ed.) pp 319-347, 2014 Wiley, N.Y. March, Advanced Organic Chemistry, fourth edition, New York: John Wiley and Sons (1992), Chapters 10 to 16; Carey and Sundberg, Advanced Organic Chemistry, Part B, Plenum (1990), Chapters 1-11; and Coltman et al., Principles and Applications of Organotransition Metal Chemistry, University Science Books, Mill Valley, Calif. (1987), Chapters 13 to 20; each of which is incorporated herein by reference in its entirety.

### Methods of Synthesizing a DNA-Encoded Compound Using a Precursor Molecule

The invention provides methods of synthesizing DNA-encoded compounds using a precursor molecule including an initial building block and a DNA oligonucleotide. The precursor molecules are prepared by methods involving reverse transcription of the DNA oligonucleotide from an RNA template.

In the provided methods, the use of reverse transcription allows for the generation of a precursor molecule including a DNA oligonucleotide and an initial building block, or a reactive moiety which can accept the initial building block, from an RNA molecule template and a charged or uncharged carrier. In these methods, reverse transcription alleviates the need to sort the precursor molecules to hybridization arrays to install the initial building blocks. Instead, the initial building block (or a reactive moiety which can accept the initial building block) are introduced by the charged or uncharged carrier, and the codons encoding the addition of the positional building blocks are added to the carriers from an RNA template to form the precursor molecule.

### Forming the Precursor Molecule

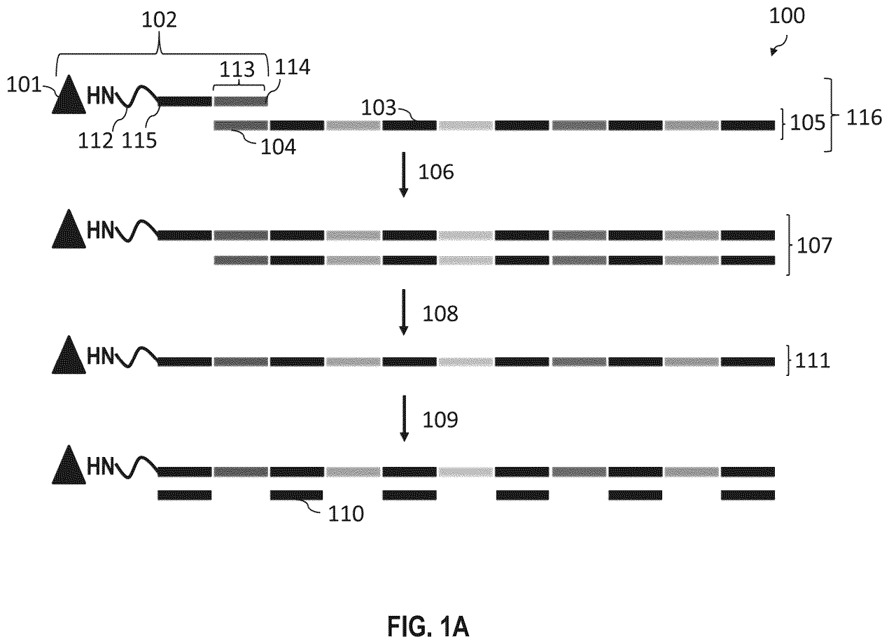

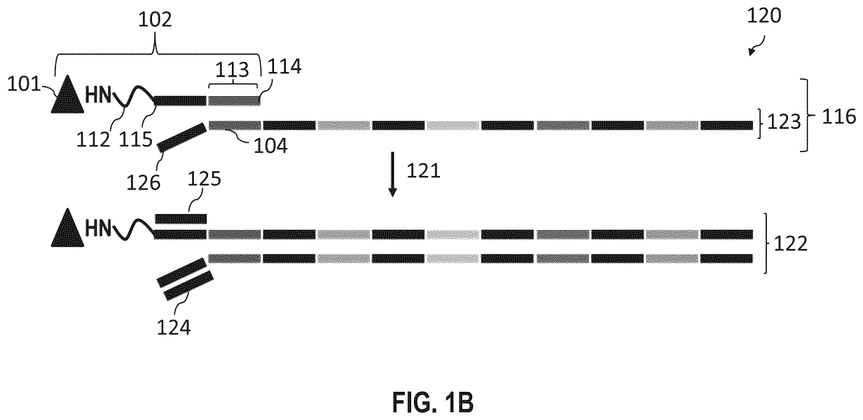

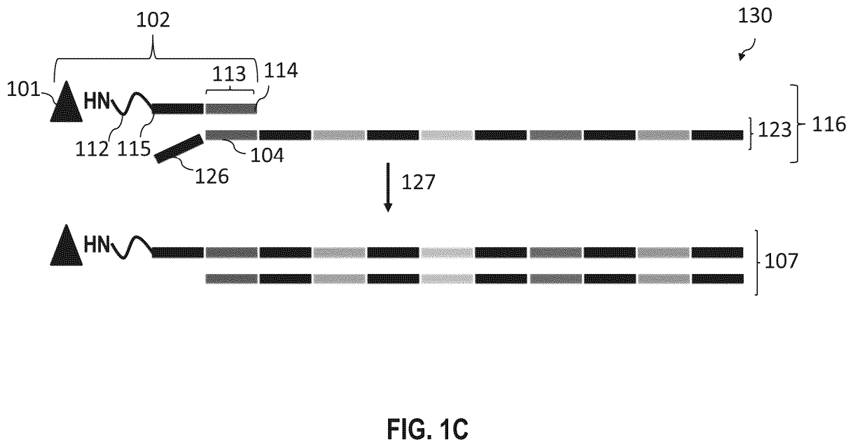

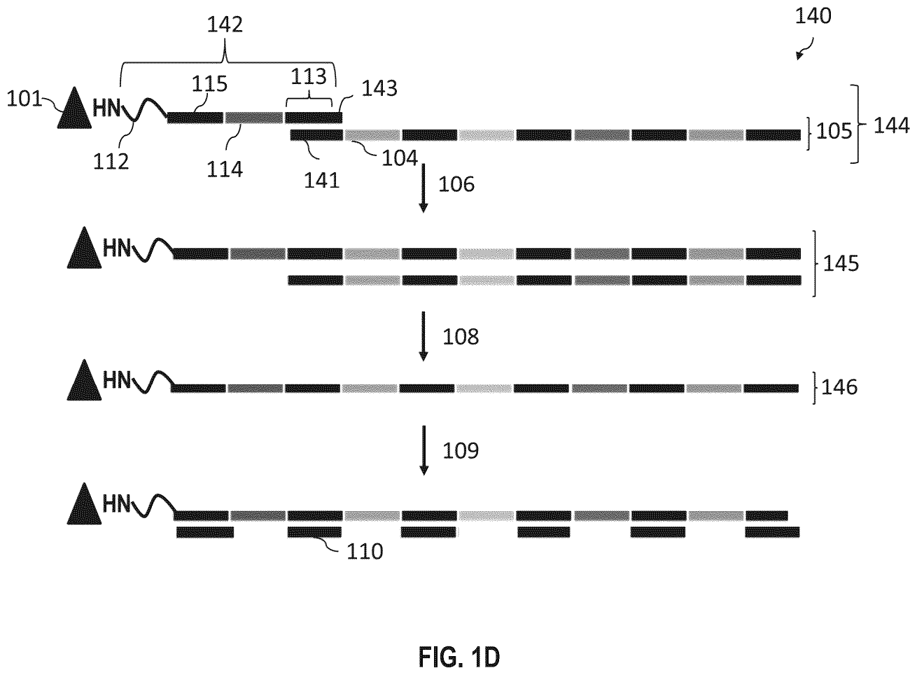

DNA-encoded compounds may be synthesized using a precursor molecule and at least one positional building block. The precursor molecules include at least one initial building block and a DNA oligonucleotide. Several alternative methods are described for forming a precursor molecule compatible for use in a method of synthesizing a DNA-encoded compound. Exemplary methods of forming a precursor according to the present disclosure are shown in FIGS. 1A-4.

FIG. 1A shows an exemplary method of forming a precursor molecule 111 for use in a method of synthesizing a DNA-encoded compound, where the precursor molecule is formed using a charged carrier 102. The charged carrier 102 includes an initial building block 101 (arbitrarily represented as a triangle with a chemical bond involving nitrogen (e.g., HN)) connected by a linker 112 to an oligonucleotide sequence including a complementary region 113 that is complementary to a portion on an RNA molecule 105. The oligonucleotide sequence including the complementary region 113 includes a codon 114 that identifies and corresponds to the initial building block 101, and an optional non-coding region 115. The oligonucleotide sequence including the complementary region 113 may include additional non-coding regions (such as 115). In the exemplary charged carrier 102, the initial building block 101 is connected by a linker 112 to a non-coding region 115 of the oligonucleotide sequence including the complementary region 113. However, the initial building block 101 may instead by connected by a linker 112 to the codon 114. The RNA molecule 105 includes a complementary coding region including a plurality of sequences that are complementary to codons (such as 104) and optional non-coding regions (such as 103). The RNA molecule 105 may include additional non-coding regions (such as 103). The sequence 104 on the RNA molecule 105 is complementary to the codon 114 on the charged carrier 102. Other complementary sequences of codons (such as 104) identify and correspond to building blocks (that is, positional building blocks) of a DNA-encoded compound from the precursor molecule 111.

The method of forming a precursor molecule in FIG. 1A begins with providing the charged carrier 102 and the RNA molecule 105. At 100, the complementary region 113 of the charged carrier 102 hybridizes to the RNA molecule 105. Specifically, the codon 114 of the complementary region 113 of the charged carrier hybridizes to the complementary sequence 104 on the RNA molecule 105 to form a charged RNA molecule 116. The charged RNA molecule 116 may be reverse transcribed at step 106 to form a DNA-RNA heteroduplex 107. RNA hydrolysis may be subsequently performed on the DNA-RNA heteroduplex at step 108 to form the precursor molecule 111. Optionally, blocking oligonucleotides 110 may be hybridized to at least one of the non-coding regions (such as 103) of the precursor molecule 111, such as to prevent self-hybridization of the precursor molecule 111 or inter-strand hybridization with other precursor molecules.

FIGS. 1B and 1C show exemplary schemes of the initial steps of forming a precursor molecule 111 for use in a method of synthesizing a DNA-encoded compound, where the precursor molecule is formed using a charged carrier 102 and the RNA molecule 123 includes an extra non-coding region 126. This extra non-coding region is not complementary to the non-coding region 115 on the charged carrier 102.

As shown in FIG. 1B, the method of forming a precursor molecule can begin with charged carrier 102 and the RNA molecule 123, which includes an extra non-coding region 126 at its terminus. At 120, the complementary region 113 of the charged carrier 102 hybridizes to the RNA molecule 123. Specifically, the codon 114 of the complementary region 113 of the charged carrier hybridizes to the complementary sequence 104 on the RNA molecule 123 to form a charged RNA molecule 122. Blocking oligonucleotide 124 may be hybridized to the extra non-coding region 126 at the terminus of the charged RNA molecule 122 and blocking oligonucleotide 125 may be hybridized to the non-coding region 115 of the charged carrier to form a DNA-RNA heteroduplex 122. Although two blocking oligonucleotide (124 and 125) are shown in FIG. 1B, it should be understood that this is a representative embodiment. Alternatively, only blocking oligonucleotide 124 may hybridized to the extra non-coding region 126 at the terminus of the charged RNA molecule 122 without the need for blocking oligonucleotide 125. The reverse is also possible, whereby only blocking oligonucleotide 125 may hybridized to the non-coding region 115 of the charged carrier 102 without the need for blocking oligonucleotide 124. Once one or both of the non-coding regions (i.e., non-coding region 115 or non-coding region 126) are hybridized to blocking oligonucleotides, the method of forming a precursor molecule can proceed as illustrated in FIG. 1A.

FIG. 1C shows yet another alternative route of forming a precursor molecule when the RNA molecule 123 includes an extra non-coding region 126 at its terminus, according to the provided embodiments. At 130, the complementary region 113 of the charged carrier 102 hybridizes to the RNA molecule 123. Specifically, the codon 114 of the complementary region 113 of the charged carrier hybridizes to the complementary sequence 104 on the RNA molecule 123 to form a charged RNA molecule 122. In this embodiment, the extra non-coding region 126 at the terminus of the RNA molecule 123 can be removed, optionally by restriction digestion, at 127, thereby leaving form a DNA-RNA heteroduplex 107. Once this digestion reaction is complete, the method of forming a precursor molecule can proceed as illustrated in FIG. 1A.

The techniques illustrated in FIGS. 1B and 1C may be utilized in any of the iterations of the methods of forming a precursor molecule provided herein, where the RNA molecule includes an extra non-coding region at its terminus.

FIG. 1D shows an alternative route of the exemplary method of forming a precursor molecule 111 for use in a method of synthesizing a DNA-encoded compound, where the precursor molecule is formed using a charged carrier 142. Here, the charged carrier 102 includes an initial building block 101 (arbitrarily represented as a triangle with an ‘NH’ bond) connected by a linker 112 to an oligonucleotide sequence including a complementary region 113 that is complementary to a portion on an RNA molecule 105. The oligonucleotide sequence including the complementary region 113 includes a non-coding region 115, a conserved non-coding region 143, and a codon 114 that identifies and corresponds to the initial building block 101. The oligonucleotide sequence including the complementary region 113 may include additional non-coding regions (such as 115) that are not complementary to the RNA molecule 105. In the exemplary charged carrier 142, the initial building block 101 is connected by a linker 112 to a non-coding region 115 of the charged carrier 142. The RNA molecule 105 includes a conserved non-coding region 141 that is complementary to the conserved non-coding region 143 of the charged carrier 102, as well as coding regions (such as 104) and other non-coding regions. Other complementary sequences of codons (such as 104) identify and correspond to building blocks (that is, positional building blocks) of a DNA-encoded compound from the precursor molecule 146.

The method of forming a precursor molecule in FIG. 1D begins with providing the charged carrier 102 and the RNA molecule 105. At 140, the complementary region 113 of the charged carrier 102 hybridizes to the RNA molecule 105. Specifically, the conserved non-coding region 143 of the complementary region 113 of the charged carrier hybridizes to a conserved complementary sequence 141 on the RNA molecule 105 to form a charged RNA molecule 144. The charged RNA molecule 144 may be reverse transcribed at step 106 to form a DNA-RNA heteroduplex 145. RNA hydrolysis may be subsequently performed on the DNA-RNA heteroduplex at step 108 to form the precursor molecule 146. Optionally, blocking oligonucleotides 110 may be hybridized to at least one of the non-coding regions (such as 103) of the precursor molecule 146, such as to prevent self-hybridization of the precursor molecule 146 or inter-strand hybridization with other precursor molecules.

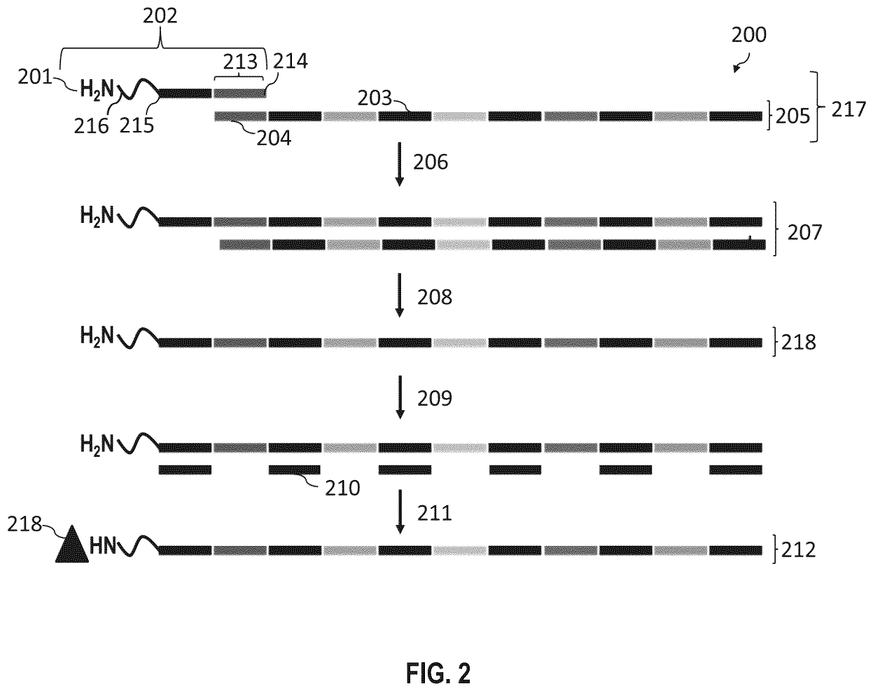

FIG. 2 shows an additional exemplary method of forming a precursor molecule for use in a method of synthesizing a DNA-encoded compound, where the precursor molecule is formed using an uncharged carrier 202. The uncharged carrier 202 includes a reactive moiety 201 (arbitrarily represented as an amine group) connected by a linker 216 to an oligonucleotide sequence including a complementary region 213 that is complementary to a portion on an RNA molecule 205. The oligonucleotide sequence including the complementary region 213 includes a codon 214 that identifies and corresponds to an initial building block 218, and an optional non-coding region such as 215. The oligonucleotide sequence including the complementary region 213 may include additional non-coding regions (such as 215). In the exemplary uncharged carrier 202, the reactive moiety 201 is connected by a linker 216 to a non-coding region 215 of the oligonucleotide sequence including the complementary region 213. However, the reactive moiety 201 may instead by connected by a linker 216 to the codon 214. The RNA molecule 205 includes a complementary coding region including a plurality of sequences that are complementary to codons (such as 204) and optional non-coding regions (such as 203). The RNA molecule 205 may include additional non-coding regions (such as 203). The sequence 204 on the RNA molecule 205 is complementary to the codon 214 on the uncharged carrier 202. Other complementary sequences of codons (such as 204) identify and correspond to building blocks (e.g., positional building blocks) useful for the synthesis of a DNA-encoded compound from the precursor molecule 212.

The method of forming a precursor molecule in FIG. 2 begins with providing the uncharged carrier 202 and the RNA molecule 205. At 200, the complementary region 213 of the uncharged carrier 202 hybridizes to the RNA molecule 205. Specifically, the codon 214 of the complementary region 213 of the charged carrier hybridizes to the complementary sequence 204 on the RNA molecule 205 to form an uncharged RNA molecule 217. The uncharged RNA molecule 217 may be reverse transcribed to form a DNA-RNA heteroduplex 207, shown in step 206. RNA hydrolysis may be subsequently performed on the DNA-RNA heteroduplex, shown in step 208, to form a DNA molecule including a reactive moiety at or near its 5′ terminus. Optionally, blocking oligonucleotides 211 may be hybridized to at least one of the non-coding regions (such as 203) to prevent self-hybridization. Finally, an initial building block 218 is reacted with the reactive moiety 201 to form the precursor molecule 212.

In some embodiments, the method of synthesizing a DNA-encoded compound includes forming a precursor molecule including a DNA oligonucleotide and an initial building block. In some embodiments, the precursor molecule includes the initial building block at or near its 5′ terminus. In some embodiments, the precursor molecule includes a first initial building block at or near its 5′ terminus and a second initial building block at or near its 3′ terminus. In some embodiments, the first initial building block at or near the 5′ terminus of the precursor molecule is the same as the second initial building block at or near the 3′ terminus of the precursor molecule. In some embodiments, the first initial building block at or near the 5′ terminus is added to the precursor molecule prior to the second initial building block at or near the 3′ terminus of the precursor molecule. In some embodiments, the first initial building block at or near the 5′ terminus is added to the precursor molecule simultaneously with the second initial building block at or near the 3′ terminus of the precursor molecule. In some embodiments, the initial building block is not a nucleic acid or nucleic acid analog. In some embodiments, the second initial building block is not a nucleic acid or nucleic acid analog. In some embodiments, the initial building block is attached to the precursor molecule by a non-nucleotide linker. In some embodiments, the second initial building block is attached to the precursor molecule by a non-nucleotide linker. In some embodiments, the precursor molecule further includes at least one non-coding region.

In some embodiments, the method of synthesizing a DNA-encoded compound includes forming a precursor molecule including a DNA oligonucleotide and an initial building block, wherein the method includes providing a carrier and an RNA molecule.

In some embodiments, method includes providing a charged carrier. In some embodiments, the charged carrier includes the initial building block and an oligonucleotide sequence including a complementary region that is complementary to a portion on the RNA molecule. In some embodiments, the oligonucleotide sequence including the complementary region includes a codon that identifies the initial building block. In some embodiments, the region that is complementary to a portion of the RNA molecule contains the codon. In some embodiments, the region that is complementary to a portion of the RNA molecule contains a conserved non-coding region. In some embodiments, the complementary region of the RNA molecule contains a complementary sequence of the codon. In some embodiments, the complementary region of the RNA molecule contains a complementary sequence of the conserved non-coding region. In some embodiments, the codon on the charged carrier hybridizes to the complementary sequence in the complementary region of the RNA molecule. In some embodiments, the conserved non-coding region on the charged carrier hybridizes to the complementary sequence in the complementary region of the RNA molecule.

In some embodiments, the method includes providing an uncharged carrier. In some embodiments, the uncharged carrier includes a reactive moiety and an oligonucleotide sequence including a complementary region that is complementary to a portion on the RNA molecule. In some embodiments, the region that is complementary to a portion of the RNA molecule contains the codon. In some embodiments, the region that is complementary to a portion of the RNA molecule contains a conserved non-coding region. In some embodiments, the complementary region of the RNA molecule contains a complementary sequence of the codon. In some embodiments, the complementary region of the RNA molecule contains a complementary sequence of the conserved non-coding region. In some embodiments, the codon on the uncharged carrier hybridizes to the complementary sequence in the complementary region of the RNA molecule. In some embodiments, the conserved non-coding region on the uncharged carrier hybridizes to the complementary sequence in the complementary region of the RNA molecule.

In some embodiments, the initial building block is not a nucleic acid or nucleic acid analog. In some embodiments, the initial building block is attached to the precursor molecule by a non-nucleotide linker. In some embodiments, the precursor molecule further includes at least one non-coding region, additional codons, a conserved non-coding region, or any combination thereof.

The charged carrier or uncharged carrier is hybridized to the RNA molecule. In the embodiments where the RNA molecule includes a non-coding region at its 5′ terminus, the non-coding region may be blocking using blocking nucleotides or digested by restriction digestion. In some embodiments, the RNA molecule includes a non-coding region at its 5′ terminus. In some embodiments, the non-coding region at the 5′ terminus of the RNA molecule includes a sequence targeted by a restriction digestion enzyme (e.g., a restriction site). In some embodiments, the RNA molecule primed by the carrier (e.g., a charged carrier or an uncharged carrier) is subjected to restriction digestion to remove the non-coding region at its 5′ terminus. In some embodiments, the non-coding region at the 5′ terminus of the RNA molecule includes a sequence that is complementary to a blocking oligonucleotide. In some embodiments, the RNA molecule primed by the carrier is hybridized with blocking oligonucleotides. In some embodiments, the blocking oligonucleotides hybridize to the non-coding region at the 5′ terminus of the RNA molecule primed by the carrier. In some embodiments, the blocking oligonucleotides hybridize to a non-coding region on the carrier that is hybridized to the RNA molecule. In some embodiments, the blocking oligonucleotides hybridize to both the non-coding region at the 5′ terminus of the RNA molecule primed by the carrier and a non-coding region on the carrier that is hybridized to the RNA molecule.

The RNA molecule primed by the carrier (e.g., a charged carrier or an uncharged carrier) is reverse transcribed to form a DNA-RNA heteroduplex. After forming the DNA-RNA heteroduplex, the RNA can be degraded by RNA hydrolysis to form the precursor molecule. In some embodiments, the RNA hydrolysis includes breaking phosphodiester bonds in the sugar-phosphate backbone of the RNA molecule of the DNA-RNA heteroduplex to cleave the RNA molecule. In some embodiments, the cleaving of the RNA molecule destroys the RNA molecule of the DNA-RNA heteroduplex, resulting in a single-stranded DNA molecule including the initial building block at or near its 5′ terminus (e.g., the precursor molecule). In some embodiments, the precursor molecule is a single-stranded DNA molecule including an initial building block at or near its 5′ terminus. In some embodiments, the initial building block is not a nucleic acid or nucleic acid analog.

In some embodiments, the method of synthesizing a DNA-encoded compound includes forming a precursor molecule including a DNA oligonucleotide and an initial building block at or near its 5′ terminus, wherein the method includes providing a charged carrier. In some embodiments, the charged carrier includes the initial building block and an oligonucleotide sequence including a complementary region that is complementary to a portion on an RNA molecule. In some embodiments, the oligonucleotide sequence including the complementary region includes a codon that identifies the initial building block. The initial building block may include any of the initial building blocks described herein. In some embodiments, forming the precursor molecule includes: (a) providing a charged carrier, wherein the charged carrier includes the initial building block and an oligonucleotide sequence including a complementary region that is complementary to a portion on an RNA molecule, wherein the oligonucleotide sequence including the complementary region includes a codon that identifies the initial building block, (b) hybridizing the complementary region of the charged carrier to the RNA molecule, (c) reverse transcribing the RNA molecule primed by the charged carrier to form a DNA-RNA heteroduplex, and (d) performing RNA hydrolysis on the DNA-RNA heteroduplex to form the precursor molecule. In some embodiments, the precursor molecule further includes at least one non-coding region. In some embodiments, the method further includes hybridizing a blocking oligonucleotide to the at least one non-coding region, wherein the blocking oligonucleotide does not hybridize to the codon. In some embodiments, the initial building block is not a nucleic acid or nucleic acid analog. In some embodiments, the initial building block is attached to the precursor molecule by a non-nucleotide linker.

In some embodiments, the method of synthesizing a DNA-encoded compound includes forming a precursor molecule including a DNA oligonucleotide and an initial building block at or near its 5′ terminus, wherein the method includes providing an uncharged charged carrier. When an uncharged carrier is used, it is necessary to add the initial building block after reverse transcription. The uncharged carrier includes a reactive moiety and an oligonucleotide sequence including a complementary region that is complementary to a portion on an RNA molecule. The oligonucleotide sequence including the complementary region includes a codon that identifies the initial building block to be added. In some embodiments, the reactive moiety is reacted with the initial building block to form the precursor molecule. The initial building block may include any of the initial building blocks provided herein. In some embodiments, forming the precursor molecule includes: (a) providing an uncharged carrier, wherein the uncharged carrier includes a reactive moiety and an oligonucleotide sequence including a complementary region that is complementary to a portion on an RNA molecule, wherein the oligonucleotide sequence including the complementary region includes a codon that identifies the initial building block, (b) hybridizing the complementary region of the uncharged carrier to the RNA molecule, (c) reverse transcribing the RNA molecule primed by the uncharged carrier to form a DNA-RNA heteroduplex, (d) performing RNA hydrolysis on the DNA-RNA heteroduplex to form a DNA molecule including the reactive moiety, and (e) reacting the reactive moiety with the initial building block to form the precursor molecule. In some embodiments, the precursor molecule further includes at least one non-coding region. In some embodiments, the method further includes hybridizing a blocking oligonucleotide to the at least one non-coding region, wherein the blocking oligonucleotide does not hybridize to the codon. In some embodiments, the initial building block is not a nucleic acid or nucleic acid analog. In some embodiments, the initial building block is attached to the precursor molecule by a non-nucleotide linker.

According to some embodiments of the provided methods, a precursor molecule may be a bivalent precursor molecule. A “bivalent precursor molecule” includes a plurality of initial building blocks. The bivalent precursor molecule may include multiple copies of the same initial building block, or the bivalent precursor molecule may include, for example, two different initial building blocks. Each initial building block (e.g., a first initial building block and a second initial building block) is at or near the 3′ terminus or the 5′ terminus of the bivalent precursor molecule. For example, a first initial building block may be at the 3′ terminus of the bivalent precursor molecule, and a second initial building block may be at the 5′ terminus of the bivalent precursor molecule. The bivalent precursor molecules can be formed using a charged carrier or an uncharged carrier.

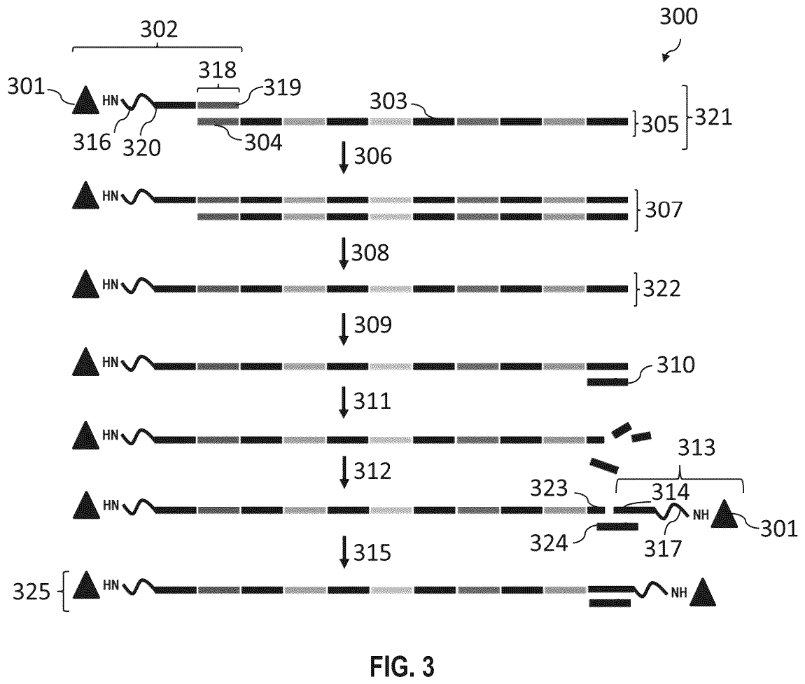

An exemplary embodiment of a method of forming a bivalent precursor molecule 325 for use in the synthesis of a DNA-encoded compound, using two charged carriers 302 and 313, is shown in FIG. 3. The charged carrier 302 includes an initial building block 301 (arbitrarily represented as a triangle with a chemical bond involving nitrogen (e.g., HN)) connected by a linker 316 to an oligonucleotide sequence including a complementary region 318 that is complementary to a portion on an RNA molecule 305. The oligonucleotide sequence including the complementary region 318 includes a codon 319 that identifies and corresponds to the initial building block 301, and an optional non-coding region such as 320. The oligonucleotide sequence including the complementary region 318 may include additional non-coding regions (such as 320). In the exemplary charged carrier 302, the initial building block 301 is connected by a linker 316 to a non-coding region 320 of the oligonucleotide sequence including the complementary region 318. However, the initial building block 301 may instead be connected by a linker 316 to the codon 319. The RNA molecule 305 includes a complementary coding region including a plurality of sequences that are complementary to codons (such as 304) and optional non-coding regions (such as 303). The RNA molecule 305 may include additional non-coding regions (such as 303). The sequence 304 on the RNA molecule 305 is complementary to the codon 319 on the charged carrier 302. Other complementary sequences of codons (such as 304) identify and correspond to building blocks (e.g., positional building blocks) useful for the synthesis of a DNA-encoded compound from the precursor molecule 322 and/or the bivalent precursor molecule 325.

The method of forming a precursor molecule in FIG. 3 begins with providing the charged carrier 302 and the RNA molecule 305. At 300, the complementary region 318 of the charged carrier 302 hybridizes to the RNA molecule 305 to form a RNA molecule 321 primed by the charged carrier. The RNA molecule 321 is reverse transcribed at step 306 to form a DNA-RNA heteroduplex 307. RNA hydrolysis at step 308 is performed on the DNA-RNA heteroduplex to form the precursor molecule 322. Optionally, blocking oligonucleotides may be hybridized to at least one of the non-coding regions (such as the non-coding region 303) of the precursor molecule 322 to prevent self-hybridization of the precursor molecule 322 (not shown in FIG. 3). In order to form the bivalent precursor molecule 325 from the precursor molecule 322, a second initial building block 301 is added to the 3′ terminus of the precursor molecule 322. First, at step 309, a first split oligonucleotide 310 is hybridized to the 3′ terminus of the precursor molecule 322 to form a restriction site. The restriction site is digested at step 311 using a restriction enzyme, thereby truncating the precursor molecule 322 at its 3′ terminus. Any suitable restriction site/restriction enzyme combination, and corresponding restriction digestion protocol, may be used to truncate the precursor molecule 322 so long as it does not interfere with the chemistry of the initial building block 301 at the 5′ terminus of the precursor molecule 322. At 312 a second splint oligonucleotide 324 is associated to the 3′ terminus 323 of the truncated precursor molecule to form an overhang. Next, a second charged carrier 313 including a second initial building block 301 connected by a second linker 317 to an oligonucleotide sequence 314 that is complementary to a portion of the second splint oligonucleotide 324. The second initial building block 301 as shown in FIG. 3 is the same as the first initial building block 301 to allow for polydisplay of the encoded portion of the resulting DNA-encoded compound (e.g., the initial building block and one or more positional building blocks extending therefrom). Alternatively, an uncharged carrier may be used rather than a charged carrier with an additional step of reacting the reactive moiety of the uncharged carrier with an individual second initial building block. In some embodiments, the second initial building block is different than the first initial building block 301. Furthermore, the second linker 317 connecting the second initial building block 301 to the oligonucleotide of the second charged carrier 313 may be the same or different from the first linker 316 connecting the first initial building block 301 to the oligonucleotide of the first charged carrier 302. Finally, at step 315 the oligonucleotide portion 314 of the charged carrier 313 is ligated to the 3′ terminus of the truncated precursor molecule using a ligase and the second splint oligonucleotide 324 to form the bivalent precursor molecule 325 with a first initial building block 301 at or near its 5′ terminus and a second initial building block 301 at or near its 3′ terminus. Optionally, and in the alternative, the 3′ end of the second charged carrier 313 includes a hairpin structure. The second initial building block 301 may be attached to the hairpin structure via a linker 317.

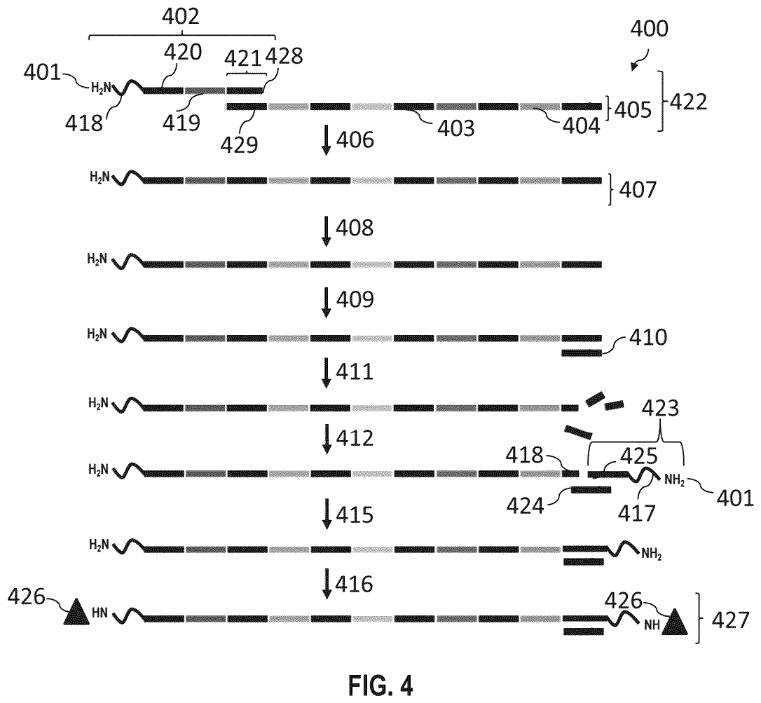

An exemplary embodiment of a method of forming a bivalent precursor molecule 427 for use in the synthesis of a DNA-encoded compound, using two uncharged carriers 402 and 423, is shown in FIG. 4. This method is similar to the method illustrated in FIG. 1D, however, it uses two uncharged carriers to form a bivalent precursor molecule. The uncharged carrier 402 includes a first reactive moiety 401 (arbitrarily represented as an amine group) connected by a linker 418 to an oligonucleotide sequence including a complementary region 421 that is complementary to a portion on an RNA molecule 405. The oligonucleotide sequence including the complementary region 421 includes a non-coding region 420, a conserved non-coding region 428, and a codon 419 that identifies and corresponds to an initial building block for reaction with the first reactive moiety 401. The oligonucleotide sequence including the complementary region 421 may include additional non-coding regions (such as 420) that are not complementary to the RNA molecule 405. In the exemplary charged carrier 402, the first reactive moiety 401 is connected by a linker 418 to a non-coding region 420 of the oligonucleotide sequence including the complementary region 421. The RNA molecule 405 includes a conserved non-coding region 429 that is complementary to the conserved non-coding region 428 of the uncharged carrier 402, as well as coding regions (such as 404) and other non-coding regions (such as 403). Other complementary sequences of codons (such as 404) identify and correspond to building blocks (e.g., positional building blocks) useful for the synthesis of a DNA-encoded compound from the bivalent precursor molecule 427.

The method of forming a precursor molecule in FIG. 4 begins with providing the uncharged carrier 402 and the RNA molecule 405. At 400, the complementary region 421 of the uncharged carrier 402 hybridizes to the RNA molecule 405. Specifically, the conserved non-coding region 428 of the complementary region 421 of the uncharged carrier hybridizes to a conserved complementary non-coding sequence 429 on the RNA molecule 405 to form an RNA molecule primed by the carrier (422). The primed RNA molecule 422 is reverse transcribed at step 406 to form a DNA-RNA heteroduplex 407. RNA hydrolysis is performed at step 408 on the DNA-RNA heteroduplex to form a DNA molecule including the first reactive moiety. Optionally, blocking oligonucleotides may be hybridized to at least one of the non-coding regions (such as 403) of the DNA molecule including the first reactive moiety to prevent self-hybridization of the DNA molecule including the first reactive moiety (not shown in FIG. 4). In order to form the bivalent precursor molecule 427 from the DNA molecule including the first reactive moiety, a second oligonucleotide including a second reactive moiety 423 (e.g., a second uncharged carrier) must be added to the 3′ terminus of the DNA molecule, and then initial building blocks must be reacted with the reactive moieties. Alternatively, the second oligonucleotide may be a charged carrier including an initial building block. First at 409, a first split oligonucleotide 410 is hybridized to the 3′ terminus of the DNA molecule including the first reactive moiety to form a restriction site. The restriction site is digested at 411 using a restriction enzyme, thereby truncating the DNA molecule at its 3′ terminus to form a truncated DNA molecule. Any suitable restriction site/restriction enzyme combination, and corresponding restriction digestion protocol, may be used to truncate the DNA molecule so long as it does not interfere with the chemistry of the first reactive moiety 301 at the 5′ terminus of the DNA molecule. At 412 a second splint oligonucleotide 424 is associated to the 3′ terminus 418 of the DNA molecule to form an overhang. Next, a second uncharged carrier 423 including a second reactive moiety 401 connected by a second linker 417 to an oligonucleotide sequence 425 that is complementary to a portion of the second splint oligonucleotide 424. Optionally, the 3′ end of the second uncharged carrier 423 includes a hairpin structure. The second reactive moiety 401 may be attached to the hairpin structure via a linker 417. The second reactive moiety 401 as shown in FIG. 4 is the same as the first reactive moiety 401 to allow for polydisplay of the encoded portion of the resulting DNA-encoded compound (e.g., the initial building block and one or more positional building blocks extending therefrom). In some embodiments, the second reactive moiety is different than the first reactive moiety 401. Furthermore, the second linker 417 connecting the second reactive moiety 401 to the oligonucleotide of the second uncharged carrier 423 may be the same or different from the first linker 416 connecting the first reactive moiety 401 to the oligonucleotide of the first uncharged carrier 402. At step 415 the oligonucleotide portion 425 of the uncharged carrier 423 is ligated to the 3′ terminus of the truncated DNA molecule using a ligase and the second splint oligonucleotide 424 to form a DNA molecule including a first reactive moiety 401 at or near its 5′ terminus and a second reactive moiety 401 at or near its 3′ terminus. Optionally, the 3′ end of the second uncharged carrier 413 includes a hairpin structure. Finally, at step 416 initial building blocks 426 are reacted with the first reactive moiety 401 at or near the 5′ terminus of the DNA molecule and the second reactive moiety 401 at or near the 3′ terminus of the DNA molecule to form the bivalent precursor molecule 427.

In some embodiments, the method of synthesizing a DNA-encoded compound includes forming a bivalent precursor molecule including a DNA oligonucleotide, an initial building block at or near its 5′ terminus, and a second initial building block at or near its 3′ terminus. In some embodiments, the method includes forming a bivalent precursor molecule including an initial building block at or near its 5′ terminus from a charged carrier and a second initial building block at or near its 3′ terminus from a charged carrier. In some embodiments, the method includes forming a bivalent precursor molecule including an initial building block at or near its 5′ terminus from a charged carrier and a second initial building block at or near its 3′ terminus from an uncharged carrier. In some embodiments, the method includes forming a bivalent precursor molecule including an initial building block at or near its 5′ terminus from an uncharged carrier a second initial building block at or near its 3′ terminus from an uncharged carrier. In some embodiments, the method includes forming a bivalent precursor molecule including an initial building block at or near its 5′ terminus from an uncharged carrier and a second initial building block at or near its 3′ terminus from a charged carrier.

In some embodiments, the method of forming a bivalent precursor molecule includes ligating a second oligonucleotide, such as a charged carrier or an uncharged carrier, to the 3′ terminus of a DNA molecule. In some embodiments, the DNA molecule is a single-stranded DNA molecule. In some embodiments, the DNA molecule is a precursor molecule including an initial building block (e.g., a first initial building block) at or near its 5′ terminus. In some embodiments, the first initial building block is attached to the DNA molecule via a linker. In some embodiments, the DNA molecule is a DNA molecule including a reactive moiety (e.g., a first reactive moiety) at or near its 5′ terminus. In some embodiments, the first reactive moiety is attached to the DNA molecule via a linker. In some embodiments, the second oligonucleotide is a charged carrier including an initial building block at or near its 3′ terminus. In some embodiments, the second oligonucleotide is an uncharged carrier including a reactive moiety at or near its 3′ terminus. In some embodiments, when the DNA molecule is a precursor molecule including a first initial building block at or near its 5′ terminus, the second oligonucleotide may be a charged carrier including a second initial building block at or near its 3′ terminus or an uncharged carrier including a reactive moiety. In some embodiments, the first initial building block and the second initial building block are the same initial building block. In some embodiments, the first initial building block and the second initial building block are different initial building blocks. In some embodiments, when the DNA molecule includes a first reactive moiety at or near its 5′ terminus, the second oligonucleotide may be a charged carrier including an initial building block at or near its 3′ terminus or an uncharged carrier including a second reactive moiety. In some embodiments, the first reactive moiety and the second reactive moiety are the same reactive moiety. In some embodiments, the first reactive moiety and the second reactive moiety are different reactive moieties.

In some embodiments, the ligation is a ligation through enzymatic means (e.g., a ligase to perform an enzymatic ligation). In some embodiments, the ligation involves chemical ligation. In some embodiments, the ligation involves template independent ligation. In some embodiments, the ligation involves template dependent ligation. In some embodiments, the ligation is mediated by one or more splint oligonucleotides.

In some embodiments, the ligation involves enzymatic ligation. In some embodiments, the enzymatic ligation involves use of a ligase. In some aspects, the ligase used herein includes an enzyme that is commonly used to join polynucleotides together or to join the ends of a single polynucleotide. An RNA ligase, a DNA ligase, or another variety of ligase can be used to ligate two nucleotide sequences together (e.g., the termini of the linear precursor nucleic acid). Ligases include ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases include bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies), and phage ligases such as T3 DNA ligase, T4 DNA ligase, and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has a DNA-splinted DNA ligase activity. In some embodiments, the ligase is a ligase that has an RNA-splinted DNA ligase activity.

In some embodiments, the ligation involves one or more splint oligonucleotides. Splint oligonucleotides may be used to hybridize to the DNA molecule (e.g., a precursor molecule including an initial building block at or near its 5′ terminus or a DNA molecule including a reactive moiety at or near its 3′ terminus) and an incoming oligonucleotide (e.g., a second oligonucleotide), such as a charged carrier or an uncharged carrier. In some embodiments, the ligation involves two splint oligonucleotides (e.g., a first splint and a second splint). In some embodiments, the ligation includes hybridizing the first splint to the 3′ terminus of the DNA molecule to form a restriction site. In some embodiments, the first splint forms a restriction site when hybridized with the DNA molecule. The restriction site is capable of being digested during a restriction digestion reaction using its corresponding restriction enzyme. Consequently, the restriction site may allow for the truncation of the DNA molecule. In some embodiments, the ligation includes digesting the restriction site to form a truncated DNA molecule. In some embodiments, the DNA molecule is truncated at or near its 3′ terminus. In some embodiments, the second splint is ligated to the 3′ terminus of the truncated DNA molecule to form an overhang. In some embodiments, the ligation can be through any of the means of ligation provided herein. In some embodiments, second splint is hybridized to the 3′ terminus of the truncated DNA molecule to form an overhang. In some embodiments, the overhang includes an oligonucleotide sequence including a complementary region to a region on the second oligonucleotide. In some embodiments, the second oligonucleotide (e.g., a charged carrier or an uncharged carrier) hybridizes to the overhang (e.g., to the complementary region on the second oligonucleotide). In some embodiments, the second oligonucleotide is ligated to the truncated DNA molecule.

In some embodiments, where the second oligonucleotide is a charged carrier including a first initial building block, the ligation of the second oligonucleotide forms a precursor molecule (e.g., where the truncated DNA molecule includes a reactive moiety at or near its 5′ terminus). In some embodiments, the method of forming a bivalent precursor molecule includes reacting the reactive moiety at or near the 5′ terminus of the precursor molecule with a second initial building block to form the bivalent precursor molecule. In some embodiments, the first initial building block and the second initial building block are the same. In some embodiments, the first initial building block and the second initial building block are different. In some embodiments, the initial building blocks (e.g., the first initial building block and/or the second initial building block) are not nucleic acids or nucleic acid analogs.

In some embodiments, where the second oligonucleotide is a charged carrier including a second initial building block, the ligation of the second oligonucleotide forms a bivalent precursor molecule (e.g., where the truncated DNA molecule includes a first initial building block at or near its 5′ terminus). In some embodiments, the first initial building block and the second initial building block are the same. In some embodiments, the first initial building block and the second initial building block are different. In some embodiments, the initial building blocks (e.g., the first initial building block and/or the second initial building block) are not nucleic acids or nucleic acid analogs.

In some embodiments, where the second oligonucleotide is an uncharged carrier including a reactive moiety, the ligation of the second oligonucleotide forms a precursor molecule (e.g., where the truncated DNA molecule includes a first initial building block at or near its 5′ terminus). In some embodiments, the method of forming a bivalent precursor molecule includes reacting the reactive moiety at the 3′ terminus of the precursor molecule with a second initial building block to form the bivalent precursor molecule. In some embodiments, the first initial building block and the second initial building block are the same. In some embodiments, the first initial building block and the second initial building block are different. In some embodiments, the initial building blocks (e.g., the first initial building block and/or the second initial building block) are not nucleic acids or nucleic acid analogs.

In some embodiments, where the second oligonucleotide is an uncharged carrier including a second reactive moiety, the ligation of the second oligonucleotide forms a precursor molecule (e.g., where the truncated DNA molecule includes a first reactive moiety at or near its 5′ terminus). In some embodiments, the method of forming a bivalent precursor molecule includes reacting the first reactive moiety at the 5′ terminus of the truncated DNA molecule and the second reactive moiety at the 3′ terminus of the truncated DNA molecule with a first initial building block and second initial building block to form the bivalent precursor molecule. In some embodiments, the first initial building block is added to the bivalent precursor molecule, and allowed to react, prior to the second initial building block. In some embodiments, the second initial building block is added to the bivalent precursor molecule, and allowed to react, prior to the first initial building block. In some embodiments, the first initial building block and the second initial building block are added to the bivalent precursor molecule, and allowed to react, at the same time. In some embodiments, the first reactive moiety and the second reactive moiety are the same. In some embodiments, the first reactive moiety and the second reactive moiety are different. In some embodiments, the first initial building block and the second initial building block are the same. In some embodiments, the first initial building block and the second initial building block are different. In some embodiments, the initial building blocks (e.g., the first initial building block and/or the second initial building block) are not nucleic acids or nucleic acid analogs.

In some embodiments, the second oligonucleotide includes a hairpin structure. A “hairpin structure” may include oligonucleotides including a loop portion, a stem portion, and a single stranded portion. In some embodiments, the hairpin structure allows for the polydisplay of multiple encoded portions of the DNA-encoded compound produced using the precursor molecules provided herein. In some embodiments, the hairpin structure is at the 3′ end of the second oligonucleotide. In some embodiments, the hairpin structure includes an initial building block, e.g., wherein the second oligonucleotide is a charged carrier. In some embodiments, the hairpin structure includes a reactive moiety, e.g., wherein the second oligonucleotide is an uncharged carrier. In some embodiments, the hairpin structure includes one or more reactive moieties. In some embodiments, the hairpin structure includes one or more initial building blocks. In some embodiments, the hairpin structure includes one or more reactive moieties and one or more initial building blocks.

In some embodiments, the method of forming a bivalent precursor molecule includes: (A) hybridizing a first splint to the 3′ terminus of the single-stranded DNA molecule to form a restriction site; (B) digesting the restriction site to form a truncated DNA molecule; (C) hybridizing or ligating a second splint to the 3′ terminus of the truncated DNA molecule, wherein the second splint forms an overhang; (D) hybridizing the second oligonucleotide to the overhang; (E) ligating the second oligonucleotide to the truncated DNA molecule. In some embodiments, the 3′ end of the second oligonucleotide includes a hairpin structure including the second initial building block. In some embodiments, the method includes: (i) ligating a second oligonucleotide to the 3′ terminus of the DNA molecule, wherein the second oligonucleotide includes a second reactive moiety at or near its 3′ terminus; (ii) reacting the reactive moiety with the initial building block and reacting the second reactive moiety with a second initial building block to form the precursor molecule. In some embodiments, the ligating includes: (A) hybridizing a first splint to the 3′ terminus of the single-stranded DNA molecule to form a restriction site; (B) digesting the restriction site to form a truncated DNA molecule; (C) hybridizing or ligating a second splint to the 3′ terminus of the truncated DNA molecule, wherein the second splint forms an overhang; (D) hybridizing the second oligonucleotide to the overhang; (E) ligating the second oligonucleotide to the truncated DNA molecule. In some embodiments, the 3′ end of the second oligonucleotide includes a hairpin structure including the reactive moiety. In some embodiments, the initial building block is not a nucleic acid or nucleic acid analog. In some embodiments, the second initial building block is not a nucleic acid or nucleic acid analog. In some embodiments, the initial building block is attached to the precursor molecule by a non-nucleotide linker. In some embodiments, the second initial building block is attached to the precursor molecule by a non-nucleotide linker. In some embodiments, the precursor molecule further includes at least one non-coding region. In some embodiments, the method further includes hybridizing a blocking oligonucleotide to the at least one non-coding region, wherein the blocking oligonucleotide does not hybridize to the codon.

In some embodiments, the method of synthesizing a DNA-encoded compound includes forming a bivalent precursor molecule including a DNA oligonucleotide, an initial building block at or near its 5′ terminus, and a second initial building block at or near its 3′ terminus, wherein the method includes providing a charged carrier with the initial building block. In some embodiments, the charged carrier includes the initial building block and an oligonucleotide sequence including a complementary region that is complementary to a portion on an RNA molecule. In some embodiments, the oligonucleotide sequence including the complementary region includes a codon that identifies the initial building block. The initial building block may include any of the initial building blocks provided herein. In some embodiments, forming the bivalent precursor molecule includes: (a) providing a charged carrier, wherein the charged carrier includes the initial building block and an oligonucleotide sequence including a complementary region that is complementary to a portion on an RNA molecule, wherein the oligonucleotide sequence including the complementary region includes a codon that identifies the initial building block, (b) hybridizing the complementary region of the charged carrier to the RNA molecule to form a charged RNA molecule, (c) reverse transcribing the charged RNA molecule to form a DNA-RNA heteroduplex, (d) performing RNA hydrolysis on the DNA-RNA heteroduplex, and (e) ligating a second oligonucleotide to the 3′ terminus of the DNA molecule, wherein the second oligonucleotide includes a second initial building block at or near its 3′ terminus to form the bivalent precursor molecule. In some embodiments, forming the bivalent precursor molecule includes: (a) providing a charged carrier, wherein the charged carrier includes the initial building block and an oligonucleotide sequence including a complementary region that is complementary to a portion on an RNA molecule, wherein the oligonucleotide sequence including the complementary region includes a codon that identifies the initial building block, (b) hybridizing the complementary region of the charged carrier to the RNA molecule to form a charged RNA molecule, (c) reverse transcribing the charged RNA molecule to form a DNA-RNA heteroduplex, (d) performing RNA hydrolysis on the DNA-RNA heteroduplex, (e) ligating a second oligonucleotide to the 3′ terminus of the DNA molecule, wherein the second oligonucleotide includes a reactive moiety at or near its 3′ terminus, and (f) reacting the reactive moiety with a second initial building block to form the precursor molecule. In some embodiments, the ligating includes: (A) hybridizing a first splint to the 3′ terminus of the single-stranded DNA molecule to form a restriction site; (B) digesting the restriction site to form a truncated DNA molecule; (C) hybridizing or ligating a second splint to the 3′ terminus of the truncated DNA molecule, wherein the second splint forms an overhang; (D) hybridizing the second oligonucleotide to the overhang; (E) ligating the second oligonucleotide to the truncated DNA molecule. In some embodiments, the 3′ end of the second oligonucleotide includes a hairpin structure including the second initial building block or the reactive moiety. In some embodiments, the bivalent precursor molecule further includes at least one non-coding region. In some embodiments, the method further includes hybridizing a blocking oligonucleotide to the at least one non-coding region, wherein the blocking oligonucleotide does not hybridize to the codon. In some embodiments, the initial building block is not a nucleic acid or nucleic acid analog. In some embodiments, the second initial building block is not a nucleic acid or nucleic acid analog. In some embodiments, the initial building block is attached to the precursor molecule by a non-nucleotide linker. In some embodiments, the second initial building block is attached to the precursor molecule by a non-nucleotide linker.

In some embodiments, the method of synthesizing a DNA-encoded compound includes forming a bivalent precursor molecule including a DNA oligonucleotide, an initial building block at or near its 5′ terminus, and a second initial building block at or near its 3′ terminus, wherein the method includes providing an uncharged carrier including the initial building block. In some embodiments, the uncharged carrier includes a reactive moiety and an oligonucleotide sequence including a complementary region that is complementary to a portion on an RNA molecule. In some embodiments, the oligonucleotide sequence including the complementary region includes a codon that identifies the initial building block. In some embodiments, the reactive moiety is reacted with the initial building block to form the precursor molecule. The initial building block may include any of the initial building blocks provided herein. In some embodiments, forming the bivalent precursor molecule includes: (a) providing an uncharged carrier, wherein the uncharged carrier includes a reactive moiety and an oligonucleotide sequence including a complementary region that is complementary to a portion on an RNA molecule, wherein the oligonucleotide sequence including the complementary region includes a codon that identifies the initial building block, (b) hybridizing the complementary region of the uncharged carrier to the RNA molecule to form an uncharged RNA molecule, (c) reverse transcribing the uncharged RNA molecule to form a DNA-RNA heteroduplex, (d) performing RNA hydrolysis on the DNA-RNA heteroduplex to form a DNA molecule including the reactive moiety, (e) ligating a second oligonucleotide to the 3′ terminus of the DNA molecule, wherein the second oligonucleotide includes a second reactive moiety at or near its 3′ terminus, and (f) reacting the reactive moiety with the initial building block and reacting the second reactive moiety with a second initial building block to form the precursor molecule. In some embodiments, forming the bivalent precursor molecule includes: (a) providing an uncharged carrier, wherein the uncharged carrier includes a reactive moiety and an oligonucleotide sequence including a complementary region that is complementary to a portion on an RNA molecule, wherein the oligonucleotide sequence including the complementary region includes a codon that identifies the initial building block, (b) hybridizing the complementary region of the uncharged carrier to the RNA molecule to form an uncharged RNA molecule, (c) reverse transcribing the uncharged RNA molecule to form a DNA-RNA heteroduplex, (d) performing RNA hydrolysis on the DNA-RNA heteroduplex to form a DNA molecule including the reactive moiety, (e) ligating a second oligonucleotide to the 3′ terminus of the DNA molecule, wherein the second oligonucleotide includes a second initial building block at or near its 3′ terminus, and (f) reacting the reactive moiety with the initial building block to form the precursor molecule. In some embodiments, the ligating includes: (A) hybridizing a first splint to the 3′ terminus of the single-stranded DNA molecule to form a restriction site; (B) digesting the restriction site to form a truncated DNA molecule; (C) hybridizing or ligating a second splint to the 3′ terminus of the truncated DNA molecule, wherein the second splint forms an overhang; (D) hybridizing the second oligonucleotide to the overhang; (E) ligating the second oligonucleotide to the truncated DNA molecule. In some embodiments, the 3′ end of the second oligonucleotide includes a hairpin structure including the second initial building block or the reactive moiety. In some embodiments, the bivalent precursor molecule further includes at least one non-coding region. In some embodiments, the method further includes hybridizing a blocking oligonucleotide to the at least one non-coding region, wherein the blocking oligonucleotide does not hybridize to the codon. In some embodiments, the initial building block is not a nucleic acid or nucleic acid analog. In some embodiments, the second initial building block is not a nucleic acid or nucleic acid analog. In some embodiments, the initial building block is attached to the precursor molecule by a non-nucleotide linker. In some embodiments, the second initial building block is attached to the precursor molecule by a non-nucleotide linker.

### Forming a Charged Carrier

The provided methods of synthesizing a DNA-encoded compound may include forming a charged carrier. The charged carrier may be used to form the precursor molecules from which the DNA-encoded compounds are synthesizes.

When preparing a library of DNA-encoded compounds, initial complexity can be provided in the design and preparation of the charged carrier. For example, in some embodiments, a plurality of different uncharged carriers are prepared including different codons. A first species of uncharged carrier may include codon C1, while a second species of uncharged carrier may include codon C2. The C1 carriers can be immobilized on a spatially-isolated solid support or otherwise separated in solution (such as in the well of a plate) from the C2 carriers. Then, a particular building block corresponding to C1 is reacted with the C1 carrier. Likewise, a different building block corresponding to C2 is reacted with the C2 carrier. Many species of charged carriers including different initial building blocks can be prepared in this manner. In some embodiments, the different species are then pooled and use to assemble a library of precursor molecules including different initial building blocks, according to the methods described herein. This library of precursor molecules may then be used to prepare a library of DNA-encoded molecules with further complexity.

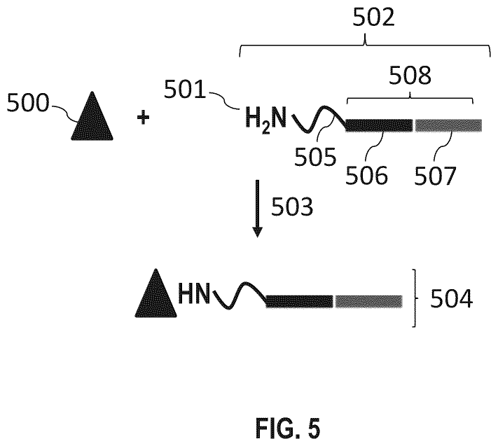

FIG. 5 illustrates an exemplary method of synthesizing a charged carrier for use in a method of synthesizing a DNA-encoded compound. An individual initial building block 500 (i.e., an initial building block that is not attached to a carrier, arbitrarily represented as a triangle) is provided along with an uncharged carrier 502. The uncharged carrier 502 includes a reactive moiety 501 (arbitrarily represented as an amine group) connected by a linker 505 to an oligonucleotide sequence including a complementary region 508 that is complementary to a portion on an RNA molecule. The oligonucleotide sequence including a complementary region 508 includes a codon 507 that identified and corresponds to the individual initial building block 500, and an optional non-coding region 506. The oligonucleotide sequence including the complementary region 508 may include additional non-coding regions (such as 506). In the exemplary uncharged carrier 502, the reactive moiety 501 is connected by a linker 505 to the non-coding region 506 of the oligonucleotide sequence including the complementary region 508. However, the reactive moiety 501 may instead by connected by a linker 505 to the codon 507.

The uncharged carrier 502 may be immobilized on a solid surface prior to step 503. Alternatively, the uncharged carrier 502 may be synthesized on a solid surface prior to step 503. At 503, the reactive moiety 501 of the uncharged carrier 502 reacts with the individual initial building block 500 to form the charged carrier 504. As described herein, the charged carrier 504 is useful for forming a precursor molecule to be used in a method of synthesizing a DNA-encoded compound.

In some embodiments, the method of synthesizing a DNA-encoded compound includes forming a charged carrier. In some embodiments, the charged carrier is formed prior to the forming of the precursor molecule. In some embodiments, the charged carrier is formed from an uncharged carrier. In some embodiments, the uncharged carrier includes a reactive moiety and an oligonucleotide sequence including a complementary region that is complementary to a portion on an RNA molecule, wherein the oligonucleotide sequence including the complementary region includes a codon that identifies an initial building block. In some embodiments, the uncharged carrier including the reactive moiety is immobilized on a solid surface. In some embodiments, the uncharged carrier including the reactive moiety is synthesized on a solid surface. In some embodiments, forming the charged carrier includes: (i) immobilizing an uncharged carrier including a reactive moiety on a solid surface; (ii) reacting the initial building block with the reactive moiety to form an immobilized charged carrier; and (iii) releasing the immobilized charged carrier from the solid support to form the charged carrier. In some embodiments, forming the charged carrier includes: (i) synthesizing an uncharged carrier including a reactive moiety on a solid surface; (ii) reacting the initial building block with the reactive moiety to form an immobilized charged carrier; and (iii) releasing the immobilized charged carrier from the solid support to form the charged carrier.

In some embodiments, the method of synthesizing a DNA-encoded compound does not include forming a charged carrier. In some embodiments, the charged carrier is formed prior to the methods provided herein. In some embodiments, the method of synthesizing a DNA-encoded compound does not involve use a charged carrier. In some embodiments, the method of synthesizing a DNA-encoded compound involves use of an uncharged carrier.

### Preparing the RNA Molecule

Synthesis of the DNA-encoded compound may further include preparing the RNA molecule including a portion (that is, an oligonucleotide sequence) that is complementary to a complementary region on an oligonucleotide sequence in a carrier. The carriers of the present disclosure (e.g., a charged carrier or an uncharged carrier) hybridize to a portion of the RNA molecules. The RNA molecules act as templates for reverse transcriptase to extend the carriers which in turn forms the oligonucleotide of the precursor molecules. The RNA molecules can then be degraded, leaving the single-stranded DNA of the precursor molecule behind, which can then optionally be made partially double stranded, such as by the addition of blocking oligonucleotides to non-coding regions.

The RNA molecule may be prepared via a polymerase chain reaction (PCR). Specifically, by using a set of PCR primers (e.g., a 5′ PCR primer and 3′ PCR primer) where at least one PCR primer of the set includes an RNA polymerase promoter sequence, a double-stranded DNA template can be amplified using PCR to form an amplified DNA template including the RNA polymerase promotor sequence. Either the 5′ PCR primer or the 3′ PCR primer, or both, may include the RNA polymerase promotor sequence, so long as at least one of the PCR primers includes the RNA polymerase promotor sequence. In some examples, the RNA polymerase promoter sequence is a T7 RNA polymerase promoter sequence. The amplified DNA template including the RNA polymerase promotor sequence can then be transcribed using an RNA polymerase to form the RNA molecule. For example, a T7 RNA polymerase can recognize the T7 RNA polymerase promoter sequence in the amplified RNA template to generate the RNA molecule.

In some examples, when the DNA template is formed by PCR, a restriction site is installed in the non-coding region at the 5′ end of the RNA molecule. After the PCR amplification, the 5′ non-coding region of the RNA molecule is removed by restriction digestion prior to transcription by T7 RNA polymerase.

In some embodiments, the method of synthesizing a DNA-encoded compound includes preparing an RNA molecule. In some embodiments, the RNA molecule includes a portion (e.g., an oligonucleotide sequence) that is complementary to a complementary region on an oligonucleotide sequence in a charged carrier. In some embodiments, the RNA molecule includes a portion (e.g., an oligonucleotide sequence) that is complementary to a complementary region on an oligonucleotide sequence in an uncharged carrier.

In some embodiments, preparing the RNA molecule includes performing PCR. In some embodiments, preparing the RNA molecule includes amplifying a double-stranded DNA template using PCR. In some embodiments, the PCR includes annealing a 5′ PCR primer and 3′ PCR primer to the double-stranded DNA template. In some embodiments, at least one of the 5′ PCR primer and the 3′ PCR primer include an RNA polymerase promoter sequence. The RNA polymerase promoter sequence may allow for the transcription of an amplified DNA template including the RNA polymerase promoter sequence to form the RNA molecule. In some embodiments, the 5′ PCR primer includes an RNA polymerase promoter sequence. In some embodiments, the 3′ PCR primer includes an RNA polymerase promoter sequence. In some embodiments, both the 5′ PCR primer and the 3′ PCR primer include an RNA polymerase promoter sequence. In some embodiments, the RNA polymerase promoter sequence is a T7 promoter sequence (e.g., a T7 RNA promoter sequence). In some embodiments, the RNA polymerase promoter sequence is recognized by an RNA polymerase. In some embodiments, the RNA polymerase is a T7 polymerase (e.g., a T7 RNA polymerase).

In some embodiments, preparing the RNA molecule includes: (a) providing a double-stranded DNA template; (b) annealing a 5′ PCR primer and 3′ PCR primer to the double-stranded DNA template, wherein at least one of the 5′ PCR primer and 3′ PCR primer include an RNA polymerase promoter sequence; (c) performing PCR to form an amplified DNA template including the RNA polymerase promoter sequence; (d) transcribing the amplified DNA template to form the RNA molecule. In some embodiments, the RNA molecule includes a portion (e.g., an oligonucleotide sequence) that is complementary to a complementary region on an oligonucleotide sequence in a charged carrier. In some embodiments, the RNA molecule includes a non-coding region at or near its 5′ terminus. In some embodiments, the RNA molecule includes a sequence that may be targeted using restriction digestion in a non-coding region at or near its 5′ terminus. In some embodiments, the RNA molecule includes a portion (e.g., an oligonucleotide sequence) that is complementary to a complementary region on an oligonucleotide sequence in an uncharged carrier. In some embodiments, the RNA polymerase promoter sequence is a T7 promoter sequence.

### Synthesizing a DNA-Encoded Compound

The precursor molecules described herein are useful for preparing (synthesizing) DNA-encoded compounds. The synthesis of the DNA-encoded compound may be encoded (e.g., directed) by the coding region of the precursor molecule.

The precursor molecule including an initial building block (or a reactive moiety which can accept the initial building block) may be used to direct the synthesis of the encoded region; or a bivalent precursor molecule including an initial building block and a second initial building block (or reactive moieties at either location), which are located at sites near opposite ends of the precursor molecule, may be used to direct the synthesis of encoded regions at either or both site(s). Thus, the DNA-encoded compound is formed from the precursor molecule. In the case of a precursor molecule with a single initial building block or reactive moiety, the encoded region includes the initial building block and at least one positional building block. In the case of a bivalent precursor molecule, a first encoded region includes a first initial building block and at least one positional building block, and a second encoded region includes a second initial building block and at least one positional building block. This system is intended to be, and by the nature of the technology are necessarily, flexible, and as such the first initial building block and the second initial building block may be the same or different and any positional building blocks may be the same or different.

After the formation of the precursor molecule (e.g., a precursor molecule including a single initial building block or a bivalent precursor molecule including two initial building blocks), a synthesized DNA-encoded compound may be formed using the precursor molecule and at least one positional building block. A variety of means of synthesizing encoded regions are well-known in the art and suitable for use with the precursor molecules described herein.

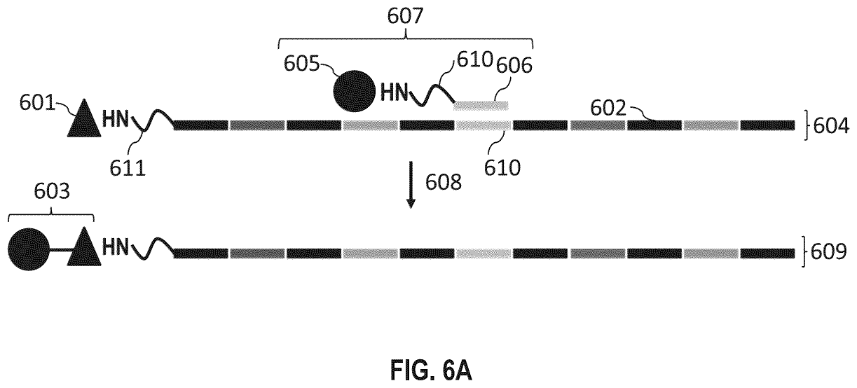

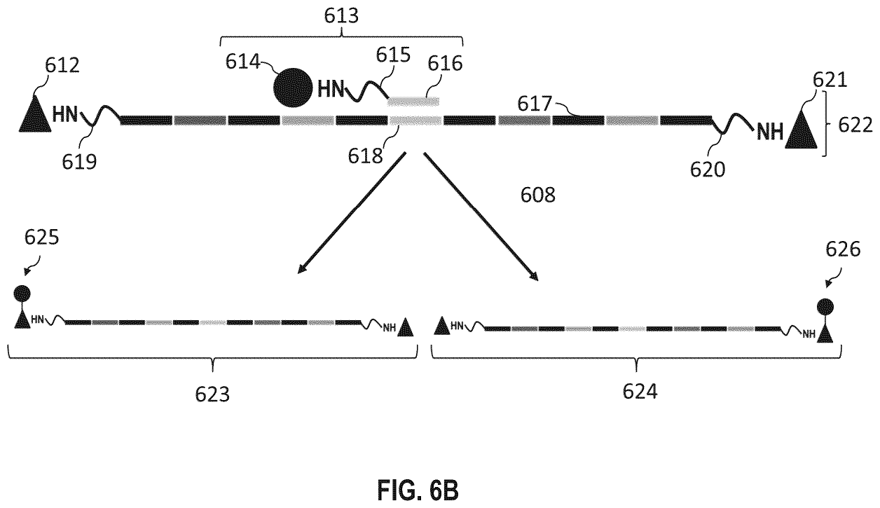

FIG. 6A shows an exemplary synthetic step (i.e., a step of adding a positional building block to the initial building block) in a method of synthesizing a DNA-encoded compound 609 using a precursor molecule 604. A charged carrier 607 (e.g., a charged anti-codon) including a positional building block 605 (arbitrarily represented as a circle) attached to an anti-codon 606 via a linker 610 hybridizes to a precursor molecule 604 is including the initial building block 601 (arbitrarily represented as a triangle) attached to a DNA oligonucleotide (e.g., coding region) via a linker 611. Specifically, the anti-codon 606 is capable of hybridizing with at least one of the codons, as shown codon 610, of the DNA oligonucleotide (e.g., coding region) of the precursor molecule 604. The anti-codon may not react with the non-coding regions (such as 602) of the precursor molecule 604. The positional building block 605 of the anti-codon 606 reacts with the initial building block 601 of the precursor molecule 604 to form a covalent bond, at 608. The reaction of the positional building block 605 with the initial building block 601 produces a synthesized DNA-encoded compound 603. The synthesized DNA-encoded compound 609 includes an initial building block 601 coupled to a positional building block 605; these two building blocks form an encoded region 603 including the synthesized compound. The process may be repeated to transfer a further positional building block to the positional building block 605 within the encoded region of the DNA-encoded compound 609. The encoded region 609 may be assessed for desirable properties, such as ability to bind a target. The building blocks of the encoded region 603 are identified by a coding region of the synthesized DNA-encoded compound 609. The positional building block 605 is exemplary and may be any suitable positional building block.

In some embodiments of the method of synthesis of a DNA-encoded compound, one or more additional charged anti-codons including additional positional building blocks hybridize to at least one of the codons of the coding region of the DNA-encoded compound, wherein the additional positional building blocks react with the positional building blocks extending from the initial building block. In some embodiments, a compound including a plurality of positional building blocks extending from the initial building is synthesized by repeating the hybridization of anti-codons and reaction of positional building blocks. In some embodiments, the synthesized encoded region extending from the initial building block does not include a nucleic acid or nucleic acid analog.

In an alternative embodiment, FIG. 6B shows an exemplary synthetic step (i.e., a step of adding a positional building block to the first and second initial building blocks) in a method of synthesizing a DNA-encoded compounds 623 and/or 624 using a bivalent precursor molecule 622. A bivalent precursor molecule 622 including a DNA molecule including a coding region including a plurality of codons, such as 618, is provided. Optionally, the bivalent precursor molecule includes one or more non-coding regions, such as 617. The bivalent precursor molecule 622 includes a first initial building block 612 (arbitrarily represented as a triangle) attached to the DNA molecule via a first linker 619 and a second initial building block 622 (arbitrarily represented as a triangle) attached to the DNA molecule via a second linker 620. As shown, the first initial building block 612 and a second initial building block 622 are the same building block. The first linker 619 and the second linker 620 may be the same linker, or they may be different linkers. A charged carrier 613 (e.g., a charged anti-codon) including an anti-codon 616 attached to a positional building block 614 (arbitrarily represented as a circle) via a linker 615 hybridizes to a codon 618 on the bivalent precursor molecule 622. At 608 a coupling reaction occurs transferring the positional building block to the first initial building block 612 or the second initial building block 621 to form a DNA-encoded compound 623 with the encoded region 625 or a DNA-encoded compound 624 with the encoded region 626. The process may be repeated to transfer a further positional building block to the positional building block 614 within the encoded region of the DNA-encoded compound 623 or 624. The encoded region 609 may be assessed for desirable properties, such as ability to bind a target. The building blocks of the encoded region, 625 or 626, are identified by a coding region of the synthesized DNA-encoded compound, 623 or 624. The positional building block 614 is exemplary and may be any suitable positional building blocks.

In an exemplary embodiment, the first encoded region and the second encoded region include an identical chemical structure. For example, if the first initial building block and the second initial building block are the same, and the type and/or order of positional building blocks attached to the first initial building block and second initial building block are the same, then the overall DNA-encoded compound will have improved binding properties for certain target molecules. In an assay designed to identify compounds that bind a target, those DNA-encoded compounds with weaker binding may be more efficiently identified when a molecule displays two or more copies of the same encoded region, as compared to a DNA-encoded compound displaying only a single copy of said encoded region.

In an additional exemplary embodiment, the first encoded region and the second encoded region include a different chemical structure. In some embodiments, the initial building block of the first encoded region is different than the second initial building block of the second encoded region. In some embodiments, the type and/or order of positional building blocks attached to the initial building block and the second initial building block are different. For example, if the initial building block and the second initial building block are different, and the type and/or order of positional building blocks attached to the first initial building block and second initial building block are different, then the total number of unique DNA-encoded compounds molecules in the DNA encoded library increases. Increasing the total number of unique DNA-encoded compound in the library similarly increases the likelihood that a molecule with desired properties will be detected (e.g., a target binding molecule). Additionally, a DNA-encoded compound including two distinct encoded regions doubles the number of synthesized compounds without increasing the number of nucleic acid strands in the system, which may be a limiting factor in the synthesis of DNA encoded libraries prepared from the DNA-encoded compounds.

In some embodiments of the method of synthesis of a DNA-encoded compound, one or more additional charged anti-codons including additional positional building blocks hybridize to at least one of the codons of the coding region of the DNA-encoded compound, wherein the additional positional building blocks react with the positional building blocks extending from the initial building block. In some embodiments, a compound including a plurality of positional building blocks extending from the initial building is synthesized by repeating the hybridization of anti-codons and reaction of positional building blocks. In some embodiments, the synthesized encoded region extending from the initial building block does not include a nucleic acid or nucleic acid analog.

### Synthesizing a DNA-Encoded Library

The DNA-encoded compounds synthesized using the methods provided herein may be used to synthesizing a DNA-encoded library. The DNA-encoded libraries include a plurality of DNA-encoded compounds formed using a plurality of precursor molecules as described herein. The plurality of precursor molecules may each include a different initial building block. The DNA-encoded library can be diverse in chemical composition, allowing for various downstream applications including selecting for encoded compounds capable of binding a target (e.g., drug discovery).

Thus, in some aspects, provided herein is a method of forming a DNA-encoded library including a plurality of DNA-encoded compounds, the method including forming a plurality of precursor molecules to synthesize the plurality of DNA-encoded compounds according to any of the provided methods, wherein each of the plurality of precursor molecules include a different initial building block. In some embodiments, the method further includes sorting the plurality of precursor molecules to a plurality of hybridization arrays, wherein, after sorting, each of the plurality of precursor molecules are further reacted with different positional building blocks corresponding to the hybridization arrays.

In some embodiments, the method of forming a DNA-encoded library includes forming a plurality of precursor molecules. In some embodiments, the plurality of precursor molecules are formed according to the methods of forming precursor molecules described herein. In some embodiments, the plurality of precursor molecules includes precursor molecules formed from a charged carrier and an RNA molecule. In some embodiments, the plurality of precursor molecules only includes precursor molecules formed from a charged carrier and an RNA molecule. In some embodiments, the plurality of precursor molecules includes precursor molecules formed from an uncharged carrier and an RNA molecule. In some embodiments, the plurality of precursor molecules only includes precursor molecules formed from an uncharged carrier and an RNA molecule. In some embodiments, the plurality of precursor molecules includes precursor molecules formed from a charged carrier and an RNA molecule and precursor molecules formed from a charged carrier and an RNA molecule.

In some embodiments, each precursor molecule of the plurality of precursor molecules includes a different initial building block. In some embodiments, the plurality of precursor molecules includes some precursor molecule including the same initial building block and some precursor molecules including a different initial building block.

In some embodiments, the method of forming a DNA-encoded library further includes sorting the plurality of precursor molecules to a plurality of hybridization arrays. A “hybridization array” includes a plurality features, wherein a feature includes a multiplicity of capture oligonucleotides (e.g., a plurality of the same capture oligonucleotides) that are capable of specifically hybridizing with a codon of the DNA oligonucleotide portion (e.g., the coding region) of a precursor molecule. To sort the plurality of precursor molecules (which each include a plurality of various codons within the DNA oligonucleotide coding region), the feature should not specifically hybridize with at least one other codon in the plurality of precursor molecules. In certain embodiments of the methods, a feature includes a substrate of at least two separate areas having immobilized capture oligonucleotides on their surface. In some embodiments, each area of the feature contains a different immobilized capture oligonucleotide, wherein the capture oligonucleotide is an oligonucleotide sequence that is capable of hybridizing with one or more codons of the coding regions of a portion of the plurality of precursor molecules. In some embodiments, the feature uses two or more chambers. In some embodiments, the chambers of the feature contain particles, such as beads, that have immobilized capture oligonucleotides on the surface of the beads.

By immobilizing a capture oligonucleotide on a feature, the plurality of precursor molecules may be sorted or selectively separated into sub-pools (i.e., portions) of precursor molecules on the basis of the particular oligonucleotide sequence of each coding region including a plurality of codons specifically hybridizing with the capture oligonucleotides. In some embodiments, the separated sub-pools of precursor molecules can then be separately released or removed from the feature into reaction chambers for further chemical processing. In some embodiments, the step of releasing is optional, not generally limited, and can include dissociating the molecules by heating, using denaturing agents, or exposing the molecules to buffer of pH≥12. In some embodiments, the chambers or areas of the hybridization array containing different immobilized oligonucleotides can be positioned to allow the contents of each chamber or area to flow into an array of wells for further chemical processing.

The feature includes a multiplicity of capture oligonucleotides (e.g., a plurality of the same capture oligonucleotides), which, in some embodiments, is particular to one feature in the system. For example, in some embodiments, a first feature includes a multiplicity of first capture oligonucleotides capable of specifically hybridizing with a plurality of codons used in a first coding position in a precursor molecule. In some embodiments, a second feature includes a multiplicity of second capture oligonucleotides capable of specifically hybridizing with a plurality of codons used in a second coding position in the precursor molecule. In some embodiments, the multiplicity of first capture oligonucleotides are different from the multiplicity of second capture oligonucleotides. In some embodiments, each feature of the system includes a different multiplicity of capture oligonucleotides from the multiplicity of capture oligonucleotides of the other features. In other words, in some embodiments, each feature is capable of specifically hybridizing with a different set of codons within a precursor molecule.

In some embodiments, the capture oligonucleotide includes between about 6 to about 50 nucleotides, such as between any of about 6 to about 20, about 8 to about 30, about 15 to about 25, and about 30 to about 50 nucleotides. In some embodiments, the anti-codon includes less than about 50 nucleotides, such as less than any of about 45, 40, 35, 30, 25, 20, 15, 10, or 6 nucleotides. In some embodiments, the anti-codon includes about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In some embodiments, the anti-codon includes between about 8 and about 30 nucleotides. In some embodiments, the length of the anti-codon is dependent on the length of the codon. In some embodiments, the length of the anti-codon is about the same as the length as the codon.

In some embodiments, the capture oligonucleotides are attached to the feature by a linker. The linker may serve to anchor the capture oligonucleotide to the feature of a hybridization array.

In some embodiments, the hybridization array includes a plurality of features (i.e., more than one feature). For example, the hybridization array may include a first feature and a second feature. In some embodiments, the hybridization array includes more than 2 features, such as any of 5, 10, 20, 30, 40, 50, 60, 70, 80, 90, 100, 150, 200, 250, 300, 350, 400, 500, 1,000, 1,500, 2,000, 2,500, 3,000, 3,500, 4,000 or more features. In some embodiments, the hybridization array includes about 96 features. In some embodiments, the hybridization array includes about 384 features. In some embodiments, the hybridization array includes about 3072 features. In some embodiments, a feature of a hybridization array includes different capture oligonucleotides than another feature of a hybridization array. In some embodiments, each feature of a hybridization array includes different capture oligonucleotides.

In some embodiments, the plurality of features of a hybridization array are electrophoretically coupled in the system. The features are “electrophoretically coupled” when charged molecules (such as precursor molecules) migrate upon application of an electric current across the system and through the features. For example, if a precursor molecule is added to a container in aqueous medium, the features of the hybridization array are electrophoretically coupled if the precursor molecule is capable of migrating across the system through each feature upon application of an electric current of sufficient voltage and for sufficient time across the system. In some embodiments, the plurality of features are connected in series (that is, in sequence) in within a system; a first feature is upstream of a second feature, a second feature is upstream of a third feature, and a third feature is upstream of a fourth feature. As a plurality of precursor molecules migrate through the system upon application of the electric current, any portion of the precursor molecules capable of specifically hybridizing with a particular feature will be immobilized at the feature and will substantially cease migrating further through the system.

In some embodiments, the features are arranged to optimize the sorting of a plurality of precursor molecules. In some embodiments, the features are substantially equidistant from one another, such that the migration time between each feature is approximately the same. In some embodiments, the features are not equidistant from one another.

In some embodiments, one or more, or all, of the features are coupled to the system such that they may be removed from the system. In some embodiments, each feature of the system may be configured to be separated from the system. In some embodiments, one feature of a plurality of features may be configured to be separated from the system, while the other features of the plurality of features may remain attached to the system. A particular feature may be separated from the system in order to separate a portion of oligonucleotides G possessing particular codons from the plurality of oligonucleotides G.

The capture of the precursor molecules by capture oligonucleotides of the hybridization array may not be 100% efficient. For example, even though a portion of precursor molecules is capable of binding capture oligonucleotides of a feature of the hybridization array, the entire portion of precursor molecules may not be completely captured by the feature. In some embodiments, substantially all of a portion of precursor molecules binds to a feature of the hybridization array including capture oligonucleotides specific for the portion of precursor molecules. In some embodiments, at least about 5% of a portion of precursor molecules binds to a feature of the hybridization array including capture oligonucleotides specific for the portion of precursor molecules, such as at least any of about 5%, 10%, 20%, 30%, 40%, 50%, 60%, 70%, 80%, 90%, 95%, 96%, 97%, 98%, 99%, and values and ranges therebetween. In some embodiments, 100% of a portion of precursor molecules binds to a feature of the hybridization array including capture oligonucleotides specific for the portion of precursor molecules.

In some embodiments, after sorting, each of the plurality of precursor molecules are further reacted with different positional building blocks corresponding to the hybridization arrays (e.g., the sequences arrayed on the hybridization arrays). In some embodiments, a positional building block is introduced to the portion of the plurality of precursor molecules on a feature. In some embodiments, the positional building block is introduced to the portion of the precursor molecules on a feature that has been separated from a hybridization array. The positional building block reacts with the portion of the precursor molecules in the encoded region of the precursor molecules (e.g., at an initial building block or a positional building block attached thereto). In some embodiments, the reaction forms a covalent bond between the plurality of precursor molecules and the positional building block.

Part or all of the coding region (e.g., the DNA molecule of the precursor molecule including a plurality of codons) of the precursor molecules may be single stranded to facilitate hybridization with the anti-codon of the charged positional building block during synthesis of an encoded molecule. In some embodiments, the hybridization of the anti-codon of the charged positional building block with the captured precursor molecules is encoded by the coding region of the precursor molecules.

It is understood that different solvents and co-reactants may be used, under acidic, basic, or neutral conditions, depending on the coupling chemistry that is used to react the charged positional building block with the initial building block of the precursor molecule.

In some embodiments, the reaction between the charged positional building block and the reactive site of the precursor molecules (e.g., precursor molecules on a feature of a hybridization array) produces a DNA-encoded library including a plurality of DNA-encoded compounds. In some embodiments, each DNA-encoded compound of the DNA-encoded library corresponds to and may be identified by the coding region of the precursor molecule. In some embodiments, the DNA-encoded compounds may be subjected to downstream analysis for selection of encoded molecules possessing specific properties (e.g., binding to a particular target molecule). The coding region of the DNA-encoded compounds selected for said properties can be PCR amplified and sequenced to determine the identity of the building blocks (e.g., the positional building blocks and initial building blocks) of the DNA-encoded compounds within the DNA-encoded library.

### EXEMPLARY EMBODIMENTS

The following embodiments are exemplary and are not intended to limit the scope of the invention or inventions described herein. Among the provided embodiments are:

Embodiment 1. A method of synthesizing a DNA-encoded compound including an initial building block and at least one positional building block, the method including:
(1) forming a precursor molecule, wherein the precursor molecule includes a DNA oligonucleotide including the initial building block at or near its 5′ terminus, wherein forming the precursor molecule includes:
   (a) providing a charged carrier, wherein the charged carrier includes the initial building block and an oligonucleotide sequence including a complementary region that is complementary to a portion on an RNA molecule, wherein the oligonucleotide sequence including the complementary region includes a codon that identifies the initial building block,
   (b) hybridizing the complementary region of the charged carrier to the RNA molecule,
   (c) reverse transcribing the RNA molecule that is primed by the charged carrier to form a DNA-RNA heteroduplex, and
   (d) performing RNA hydrolysis on the DNA-RNA heteroduplex to form the precursor molecule; and
(2) synthesizing the DNA-encoded compound using the precursor molecule and the at least one positional building block.

Embodiment 2. The method of embodiment 1, wherein:
(i) the region that is complementary to a portion of the RNA molecule contains the codon;
(ii) the complementary region of the RNA molecule contains a complementary sequence of the codon; and
(iii) the codon on the charged carrier hybridizes to the complementary sequence in the complementary region of the RNA molecule.

Embodiment 3. The method of embodiment 1, wherein:
(i) the charged carrier has the structure B-C-R, wherein B is the initial building block, R is the region that is complementary to a portion of the RNA molecule, and C is the codon that identifies the initial building block;
(ii) the complementary region of the RNA molecule does not contain a complementary sequence of the codon.

Embodiment 4. The method of any one of embodiments 1-3, further including forming the charged carrier.

Embodiment 5. The method of embodiment 4, wherein forming the charged carrier includes:
(i) immobilizing an uncharged carrier including a reactive moiety on a solid surface or synthesizing the uncharged carrier including the reactive moiety on the solid surface;
(ii) reacting the initial building block with the reactive moiety to form an immobilized charged carrier; and
(iii) releasing the immobilized charged carrier from the solid support to form the charged carrier.

Embodiment 6. A method of synthesizing a DNA-encoded compound including an initial building block and at least one positional building block, the method including:
(1) forming a precursor molecule, wherein the precursor molecule includes a DNA oligonucleotide including the initial building block at or near its 5′ terminus, wherein forming the precursor molecule includes:
   (a) providing an uncharged carrier, wherein the uncharged carrier includes a reactive moiety and an oligonucleotide sequence including a complementary region that is complementary to a portion on an RNA molecule, wherein the oligonucleotide sequence including the complementary region includes a codon that identifies the initial building block,
   (b) hybridizing the complementary region of the uncharged carrier to the RNA molecule,
   (c) reverse transcribing the RNA molecule primed by the uncharged carrier to form a DNA-RNA heteroduplex,
   (d) performing RNA hydrolysis on the DNA-RNA heteroduplex to form a DNA molecule including the reactive moiety, and
   (e) reacting the reactive moiety with the initial building block to form the precursor molecule; and
(2) synthesizing the DNA-encoded compound using the precursor molecule and the at least one positional building block.

Embodiment 7. The method of claim embodiment 6, wherein step (1)(e) includes:
(i) ligating a second oligonucleotide to the 3′ terminus of the DNA molecule, wherein the second oligonucleotide includes a second reactive moiety at or near its 3′ terminus; and
(ii) reacting the reactive moiety with the initial building block and reacting the second reactive moiety with a second initial building block to form the precursor molecule.

Embodiment 8. The method of embodiment 7, wherein step (1)(e)(i) includes:
(A) hybridizing a first splint to the 3′ terminus of the single-stranded DNA molecule to form a restriction site;
(B) digesting the restriction site to form a truncated DNA molecule;
(C) hybridizing or ligating a second splint to the 3′ terminus of the truncated DNA molecule, wherein the second splint forms an overhang;
(D) hybridizing the second oligonucleotide to the overhang; and
(E) ligating the second oligonucleotide to the truncated DNA molecule.

Embodiment 9. The method of embodiment 7 or embodiment 8, wherein the 3′ end of the second oligonucleotide includes a hairpin structure including the reactive moiety.

Embodiment 10. The method of any one of embodiments 6-9, wherein:
(i) the region that is complementary to a portion of the RNA molecule contains the codon;
(ii) the complementary region of the RNA molecule contains a complementary sequence of the codon; and
(iii) the codon on the uncharged carrier hybridizes to the complementary sequence in the complementary region of the RNA molecule.

Embodiment 11. The method of any one of embodiments 6-9, wherein:
(i) the uncharged carrier has the structure M-C-R, wherein M is the reactive moiety, C is the codon that identifies the initial building block, and R is the region that is complementary to a portion of the RNA molecule; and
(ii) the complementary region of the RNA molecule does not contain a complementary sequence of the codon.

Embodiment 12. The method of any one of embodiments 1-11, wherein the precursor molecule further includes at least one non-coding region.

Embodiment 13. The method of embodiment 12, wherein the method further includes hybridizing a blocking oligonucleotide to the at least one non-coding region, wherein the blocking oligonucleotide does not hybridize to the codon.

Embodiment 14. The method of any one of embodiments 1-13, wherein the initial building block is not a nucleic acid or nucleic acid analog.

Embodiment 15. The method of any one of embodiments 7-14, wherein the second initial building block is not a nucleic acid or nucleic acid analog.

Embodiment 16. The method of any one of embodiments 1-15, wherein the initial building block is attached to the precursor molecule by a non-nucleotide linker.

Embodiment 17. The method of any one of embodiments 7-16, wherein the second initial building block is attached to the precursor molecule by a non-nucleotide linker.

Embodiment 18. The method of any one of embodiments 1-17, wherein the method further includes preparing the RNA molecule, wherein preparing the RNA molecule includes:
(a) providing a double-stranded DNA template;
(b) annealing a 5′ polymerase chain reaction (PCR) primer and 3′ PCR primer to the double-stranded DNA template, wherein at least one of the 5′ PCR primer and Y PCR primer include an RNA polymerase promoter sequence;
(c) performing PCR to form an amplified DNA template including the RNA polymerase promoter sequence; and
(d) transcribing the amplified DNA template to form the RNA molecule.

Embodiment 19. The method of embodiment 18, wherein the RNA polymerase promoter sequence is a T7 promoter sequence.

Embodiment 20. A method of forming a DNA-encoded library including a plurality of DNA-encoded compounds, the method including forming a plurality of precursor molecules to synthesize the plurality of DNA-encoded compounds according to any one of embodiments 1-19, wherein each of the plurality of precursor molecules include a different initial building block.

Embodiment 21. The method of embodiment 20, further including sorting the plurality of precursor molecules to a plurality of hybridization arrays, wherein, after sorting, each of the plurality of precursor molecules are further reacted with different positional building blocks corresponding to the hybridization arrays.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1. Formation of a Precursor Molecule

This example illustrates a method of forming a precursor molecule by providing a charged carrier, such as according to FIG. 1A. This example further illustrates a method of synthesizing a DNA-encoded compound from the formed precursor molecule.

A double-stranded DNA template, containing relevant coding and non-coding regions was generated using primers containing coding and non-coding sequences which are stitched together using overlap extension by polymerase chain reaction (PCR). The 5′ terminal non-coding region possessed the EciI recognition sequence. The T7 promoter region was then installed within the template through a second PCR to enable transcription downstream. 7-10 nmols (across 3 sub-libraries, about 27-30 nmols total) of this T7 promoter containing amplified template was restriction digested using EciI at 37° C. overnight. This was done so that the non-coding region at the 5′ end of the template was cut, thereby leaving the first codon at the 5′ terminal end of the resulting product. This EciI digested product was then enzymatically transcribed at 37° C. overnight, cleaned up by LiCl precipitation followed by ethanol precipitation, resulting in 2,000-2,600 nmol of purified RNA molecules (across 3 sub-libraries, about 7,000 nmols total).

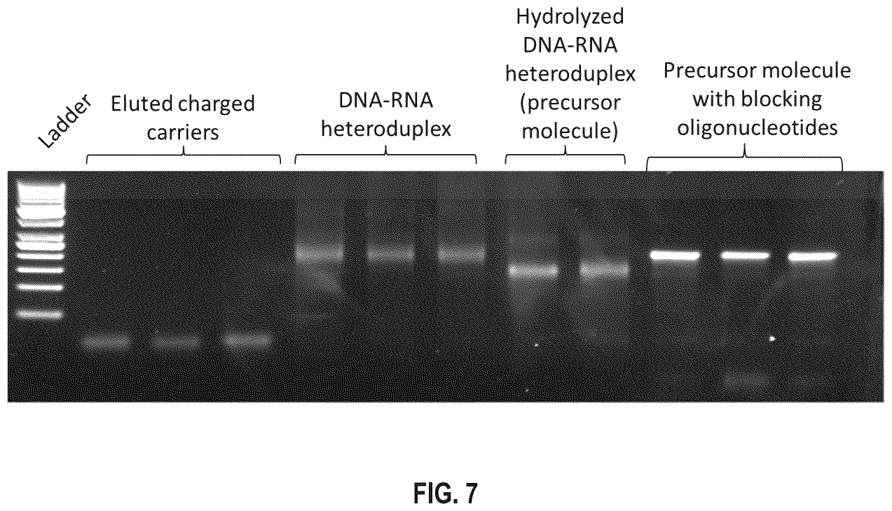

740 charged carrier codons were pooled and cleaned up by centrifuging the solution through a 3,000 Da-molecular-weight cut off filter. About 1,300-1,600 nmols (across 3 sub-libraries, about 4,300 nmols total) of charged carrier codons (shown in the left columns of FIG. 7, after the ladder) were used to prime the purified RNA. The RNA molecules were mixed with a first strand buffer to 1X concentration, and the charged carriers were hybridized to a complementary region of the RNA molecule by heating at 1.25 equivalents in the first strand buffer to 80° C. and cooling down slowly to 42° C.

Next, the RNA molecules primed by the charged carriers were reverse transcribed to form a DNA-RNA heteroduplex. Briefly, dNTPs, DTT, and RNase inhibitor were added to the RNA molecule that is primed by the charged carrier (shown in the middle-left columns of FIG. 7). The reaction mixture containing about 1,365-2,730 nmols primed RNA (across 3 sub-libraires, 4,200 nmols total) was incubated at 37° C. overnight, to form DNA-RNA heteroduplexes by reverse transcription. RNA hydrolysis was then performed on the DNA-RNA heteroduplexes using sodium hydroxide (100 mM, pH 12) at 70° C. for 15-30 minutes, to form precursor molecules with building blocks at their 5′ terminus (shown in the middle-right columns of FIG. 7). The resulting precursor molecule solution was neutralized with acetic acids.

Blocking oligonucleotides, which hybridize to at least one non-coding region of the oligonucleotide sequence of the precursor molecule, were added to the neutralized pool of precursor molecules. The blocking oligonucleotides did not hybridize to any codon of the precursor molecule. The pool of precursor molecules was cleaned using a 10,000 Da-molecular-weight cut off centrifugal filter and buffer exchanged to hybridization buffer (shown in the right columns of FIG. 7). Between about 375 and 575 nmols (across 3 sub-libraries, about 1,500 nmols total) of cleaned up, precursor molecule was obtained at the end of this process. As shown in Table 1 below, these data indicate the successful production of a high-purity precursor molecule using the methods provided herein.

TABLE 1
Precursor Molecule Yield.

| Sub- | Precursor Molecule Yield | % Yield Following Reverse- |
|---|---|---|
| Library | Following Clean Up (nmols) | Transcription (based on gel) |
| 1 | 576 | 95.1 |
| 2 | 378 | 88.4 |
| 3 | 446 | 89.9 |

A DNA-encoded compound may be synthesized using the resulting precursor molecule and at least one positional building block, according to the methods provided herein.

### Example 2. Formation of a Bivalent Precursor Molecule

This example illustrates a method of forming a bivalent precursor molecule by providing a charged carrier, such as according to FIG. 4. This example further illustrates a method of synthesizing a DNA-encoded compound from the formed bivalent precursor molecule.

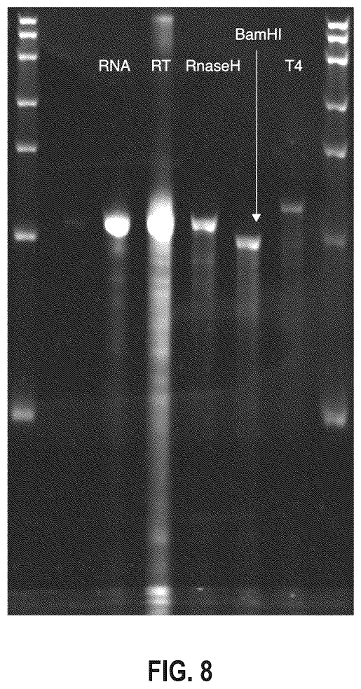

A double-stranded DNA template, containing relevant coding and non-coding regions was generated using PCR. The T7 promoter region and the BamHI restriction digestion site was then installed within the template through a second PCR to enable transcription downstream. About 30 nmols of this T7 and BamHI site containing amplified template was then enzymatically transcribed at 37° C. overnight and cleaned up by LiCl precipitation followed by ethanol precipitation, resulting in about 6,000 nmols of purified RNA molecules (shown in the first column of FIG. 8, after the ladder).

768 charged carrier codons were pooled and cleaned up by centrifuging the solution through a 3,000 Da-molecular-weight cut off filter. In each well of a deep well plate, 6 nmol of a charged carrier (containing a reactive handle/amine at the 5′ end) was used as to prime 6.6 nmol of the purified RNA with 6.6 nmol of the purified RNA. The RNA molecules were mixed with a first strand buffer to 1X concentration, and the charged carriers were hybridized to a complementary region of the RNA molecule by heating at 1.25 equivalents in the first strand buffer to 80° C. and cooling down slowly to 42° C.

Next, the RNA molecules primed by the charged carriers were reverse transcribed to form a DNA-RNA heteroduplex. Briefly, dNTPs, DTT, and RNase inhibitor were added to the RNA molecule that was primed by the charged carrier. The reaction mixture was incubated at 37° C. overnight, to form DNA-RNA heteroduplexes by reverse transcription. A total of 8 such deep well plates were used to reverse transcribe 768 carrier codons to generate the DNA-RNA heteroduplex through reverse transcription (shown in the third column of FIG. 8).

RNA hydrolysis was then performed on the DNA-RNA heteroduplexes using RNaseH at 37° C. overnight. Each well yielded between about 2.2 and 2.6 nmols of precursor molecule, determined by agarose gel and Fragment Analyzer measurements (shown in the fourth column of FIG. 8).

Next, the 3′ end of the precursor molecule was first made partially double-stranded by annealing about 3.5 nmols of a short splint to the precursor molecule, which was subsequently digested with BamHI restriction enzyme (shown in the fifth column of FIG. 8). To produce a bivalent precursor molecule, about 5 nmols of short oligonucleotides carrying a reactive handle (e.g., an amine) at their 3′ end was ligated onto the 3′ end of the BamHI digested precursor molecule, with the aid of a ligation splint. This resulted in 768 pools of codon-specific, bivalent precursor molecules (shown in the sixth column of FIG. 8).

A DNA-encoded compound may be synthesized using the resulting bivalent precursor molecule and at least one positional building block, according to the methods provided herein.

### Example 3. Synthesis of a DNA-Encoded Compound

This example illustrates a method of synthesizing a DNA encoded compound using a bivalent precursor molecule, such as a bivalent precursor molecule formed using the methods provided herein.

Briefly, initial building blocks were reacted with a reactive moiety of a bivalent precursor molecule to form a DNA-encoded compound. To immobilize the bivalent precursor molecule, bivalent precursor molecules were first bound to diethylaminoethyl (DEAE) sepharose. Specifically, 60 μL of sepharose suspension in water (1:1) was added to 1 mL of each bivalent precursor molecule's solution in water in 96-well plate. The resulting mixture was shaken until complete binding was confirmed by gel, and then filtered through a 384-well filter plate (single carrier per well, A1 quadrant wells). A mirror control oligomer on DEAE sepharose (500 pmols of oligomer, 60 μL of Sepharose suspension in water) was added to B2 quadrant wells of the plate. The resin was washed with water (3 times, 60 μL), methanol (3 times, 60 μL), 400 mM N-methyl-morpholine in dimethylacetamide (once in 60 μL), and methanol (3 times, 60 μL). The filter plate was spun down (3,000 rpm), dried with a vacuum manifold, and the bottom was sealed with a resin stopper.

Initial building blocks (200 mM solutions in DMSO) were placed in 2 copies of 96-well plates (60 μL of each initial building block). 60 μL of freshly made reagent solution (200 mM EDC-HCl, 40 mM HOAt, 400 mM N-methyl-morpholine, in ethanol) were added to each initial building block solution, mixed, and 55 μL of each initial building block solution were added to A1 quadrant wells of the filter plate and another 55 μL of each initial building block solution were added to B2 quadrant wells. The filter plate top was sealed with adhesive cover and sandwiched between metal plates that were clamped to prevent leaks. After 1 hour incubation at room temperature, the filter plate was unsealed and washed on a vacuum manifold with: DMSO:methanol (1:1, 3 times, 60 μL), methanol (3 times, 60 μL), 150 mM NaCl in water (3 times, 60 μL), methanol (3 times, 60 μL), 400 mM N-methyl-morpholine in dimethylacetamide (once, 60 μL), and methanol (3 times, 60 μL). The filter plate was spun down (3000 rpm), dried with a vacuum manifold, and the bottom was sealed with a resin stopper.

Initial building blocks in the second 96-well plate were activated with freshly made reagent solution and added to the filter plate in the same manner. The filter plate top was sealed with adhesive cover and sandwiched between metal plates that were clamped to prevent leaks. After overnight incubation at room temperature, the filter plate was unsealed and washed on a vacuum manifold with DMSO:methanol (1:1, 3 times, 60 μL), then 20% 4-methylpiperidine was added for Fmoc deprotection (10 min incubation at room temperature), and the plate was washed with DMSO:methanol (1:1, 3 times, 60 μL), methanol (3×60 μL), and 150 mM NaCl in water (3 times, 60 μL), and spun down (3000 rpm).

For DNA elution, 30 μL of 1.5 M NaCl in water (with 50 mM NaOH and 0.005% Triton X-100) were added to each well and centrifuged into a 384-well receiving plate. Elution was repeated 2 times for mirror samples and 4 times for library samples.

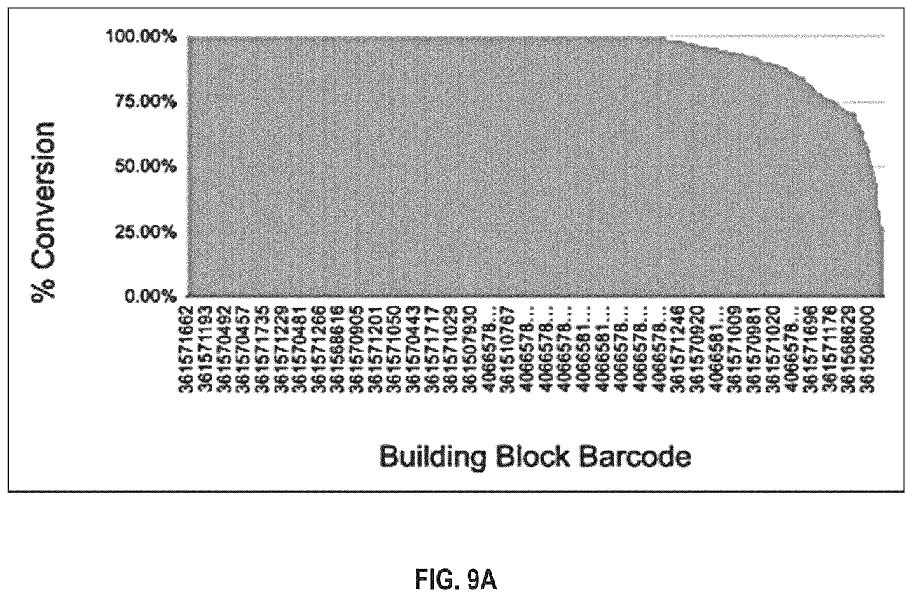

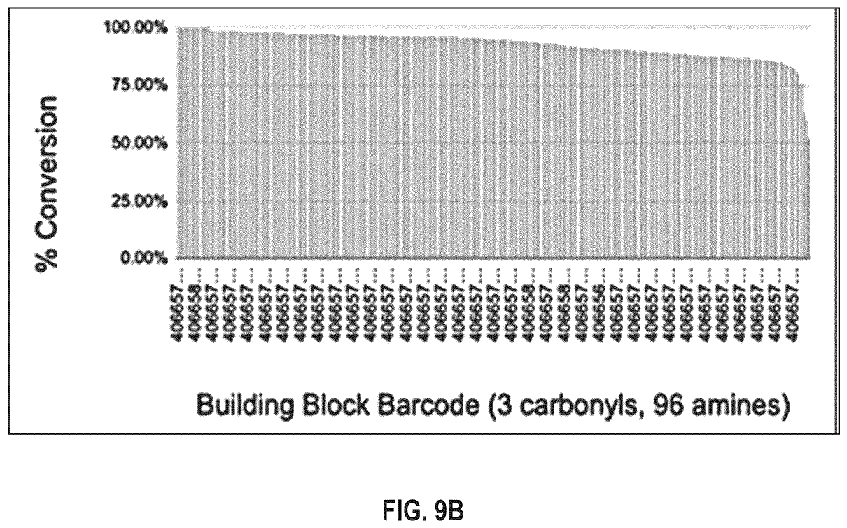

As shown in FIG. 9A, when the bivalent precursor molecules were reacted with 480 Fmoc amino acids, 472 of these reactions gave yields above 25% (Table 2).

TABLE 2
Fmoc amino acid reaction yield.

| Conversion Range | No. of Unique Synthons |
|---|---|
| 100-75% | 446 |
| 75-50% | 26 |
| 50-25% | 7 |
| 25-10% | 1 |

Similarly, FIG. 9B shows that when the bivalent precursor molecules were reacted with 288 reductoids (i.e., reactions that use reductive amination to produce a secondary amine), 285 of these reactions gave yields above 75%, with all reactions giving yields above 50% (Table 3).

TABLE 3
Reductoid reaction yield.

| Conversion Range | No. of Unique Synthons |
|---|---|
| 100-75% | 285 |
| 75-50% | 3 |
| 50-25% | 0 |
| 25-10% | 0 |

1. A method of synthesizing a DNA-encoded compound comprising an initial building block and at least one positional building block, the method comprising:
(1) forming a precursor molecule, wherein the precursor molecule comprises a DNA oligonucleotide comprising the initial building block at or near its 5′ terminus, wherein forming the precursor molecule comprises:
(a) providing a charged carrier, wherein the charged carrier comprises the initial building block and an oligonucleotide sequence comprising a complementary region that is complementary to a portion on an RNA molecule, wherein the oligonucleotide sequence comprising the complementary region comprises a codon that identifies the initial building block,
(b) hybridizing the complementary region of the charged carrier to the RNA molecule,
(c) reverse transcribing the RNA molecule that is primed by the charged carrier to form a DNA-RNA heteroduplex, and
(d) performing RNA hydrolysis on the DNA-RNA heteroduplex to form the precursor molecule; and
(2) synthesizing the DNA-encoded compound using the precursor molecule and the at least one positional building block.

2. The method of claim 1, wherein:
(i) the region that is complementary to a portion of the RNA molecule contains the codon;
(ii) the complementary region of the RNA molecule contains a complementary sequence of the codon; and
(iii) the codon on the charged carrier hybridizes to the complementary sequence in the complementary region of the RNA molecule.

3. The method of claim 1, wherein:
(i) the charged carrier has the structure B-C-R, wherein B is the initial building block, R is the region that is complementary to a portion of the RNA molecule, and C is the codon that identifies the initial building block;
(ii) the complementary region of the RNA molecule does not contain a complementary sequence of the codon.

4. The method of claim 1, further comprising forming the charged carrier.

5. The method of claim 4, wherein forming the charged carrier comprises:
(i) immobilizing an uncharged carrier comprising a reactive moiety on a solid surface or synthesizing the uncharged carrier comprising the reactive moiety on the solid surface;
(ii) reacting the initial building block with the reactive moiety to form an immobilized charged carrier; and
(iii) releasing the immobilized charged carrier from the solid support to form the charged carrier.

6. A method of synthesizing a DNA-encoded compound comprising an initial building block and at least one positional building block, the method comprising:
(1) forming a precursor molecule, wherein the precursor molecule comprises a DNA oligonucleotide comprising the initial building block at or near its 5′ terminus, wherein forming the precursor molecule comprises:
(a) providing an uncharged carrier, wherein the uncharged carrier comprises a reactive moiety and an oligonucleotide sequence comprising a complementary region that is complementary to a portion on an RNA molecule, wherein the oligonucleotide sequence comprising the complementary region comprises a codon that identifies the initial building block,
(b) hybridizing the complementary region of the uncharged carrier to the RNA molecule,
(c) reverse transcribing the RNA molecule primed by the uncharged carrier to form a DNA-RNA heteroduplex,
(d) performing RNA hydrolysis on the DNA-RNA heteroduplex to form a DNA molecule comprising the reactive moiety, and
(e) reacting the reactive moiety with the initial building block to form the precursor molecule; and
(2) synthesizing the DNA-encoded compound using the precursor molecule and the at least one positional building block.

7. The method of claim 6, wherein step (1)(e) comprises:
(i) ligating a second oligonucleotide to the 3′ terminus of the DNA molecule, wherein the second oligonucleotide comprises a second reactive moiety at or near its 3′ terminus; and
(ii) reacting the reactive moiety with the initial building block and reacting the second reactive moiety with a second initial building block to form the precursor molecule.

8. The method of claim 7, wherein step (1)(e)(i) comprises:
(A) hybridizing a first splint to the 3′ terminus of the single-stranded DNA molecule to form a restriction site;
(B) digesting the restriction site to form a truncated DNA molecule;
(C) hybridizing or ligating a second splint to the 3′ terminus of the truncated DNA molecule, wherein the second splint forms an overhang;
(D) hybridizing the second oligonucleotide to the overhang; and
(E) ligating the second oligonucleotide to the truncated DNA molecule.

9. The method of claim 7, wherein the 3′ end of the second oligonucleotide comprises a hairpin structure comprising the reactive moiety.

10. The method of claim 6, wherein:
(i) the region that is complementary to a portion of the RNA molecule contains the codon;
(ii) the complementary region of the RNA molecule contains a complementary sequence of the codon; and
(iii) the codon on the uncharged carrier hybridizes to the complementary sequence in the complementary region of the RNA molecule.

11. The method of claim 6, wherein:
(i) the uncharged carrier has the structure M-C-R, wherein M is the reactive moiety, C is the codon that identifies the initial building block, and R is the region that is complementary to a portion of the RNA molecule; and
(ii) the complementary region of the RNA molecule does not contain a complementary sequence of the codon.

12. The method of claim 1, wherein the precursor molecule further comprises at least one non-coding region.

13. The method of claim 12, wherein the method further comprises hybridizing a blocking oligonucleotide to the at least one non-coding region, wherein the blocking oligonucleotide does not hybridize to the codon.

14. The method of claim 1, wherein the initial building block is not a nucleic acid or nucleic acid analog.

15. The method of claim 1, wherein the initial building block is attached to the precursor molecule by a non-nucleotide linker.

16. The method of claim 7, wherein the second initial building block:
(i) is not a nucleic acid or nucleic acid analog; and/or
(ii) is attached to the precursor molecule by a non-nucleotide linker.

17. The method of claim 1, wherein the method further comprises preparing the RNA molecule, wherein preparing the RNA molecule comprises:
(a) providing a double-stranded DNA template;
(b) annealing a 5′ polymerase chain reaction (PCR) primer and 3′ PCR primer to the double-stranded DNA template, wherein at least one of the 5′ PCR primer and 3′ PCR primer comprise an RNA polymerase promoter sequence;
(c) performing PCR to form an amplified DNA template comprising the RNA polymerase promoter sequence; and
(d) transcribing the amplified DNA template to form the RNA molecule.

18. The method of claim 17, wherein the RNA polymerase promoter sequence is a T7 promoter sequence.

19. A method of forming a DNA-encoded library comprising a plurality of DNA-encoded compounds, the method comprising forming a plurality of precursor molecules to synthesize the plurality of DNA-encoded compounds according to claim 1, wherein each of the plurality of precursor molecules comprise a different initial building block.

20. The method of claim 19, further comprising sorting the plurality of precursor molecules to a plurality of hybridization arrays, wherein, after sorting, each of the plurality of precursor molecules are further reacted with different positional building blocks corresponding to the hybridization arrays.
